(19) United States
(12) Patent Application Publication
(10) Pub. No.: US 2026/0317837 A1
(43) Pub. Date: Oct. 15, 2026

(54) COMPOSITIONS, KITS, AND METHODS FOR DETECTION OF SYPHILIS

(71) Applicant: University of Washington, Seattle, WA (US)

(72) Inventors: Joshua A. Lieberman, Seattle, WA (US); Stephen J. Salipante, Seattle, WA (US)

(73) Assignee: University of Washington, Seattle, WA (US)

(21) Appl. No.: 19/104,801

(22) PCT Filed: Aug. 30, 2023
(86) PCT No.: PCT/US2023/073181

§ 371 Date: Feb. 19, 2025

Related U.S. Application Data
(60) Provisional application No. 63/402,787, filed on Aug. 31, 2022.

(51) Int. Cl. C12Q 1/689

(52) U.S. Cl. CPC C12Q 1/689

(57) ABSTRACT

Products, systems, compositions, kits, and methods that include or utilize site-specific primers for detection of syphilis in a sample from a patient via a loop-mediated isothermal amplification (LAMP) reaction. The site-specific primers anneal to a T. pallidum nucleic acid in the sample. The sample is contacted with a strand-displacing DNA polymerase to amplify a portion of the T. pallidum nucleic acid based on anneal positions of the site-specific primers to produce amplified products of the LAMP reaction. The amplified products, if present as a result of the LAMP reaction, are detected to determine T. pallidum is present in the sample. The disclosed elements can be used for clinical tests to determine whether the patient has syphilis as the basis for further clinical testing or treatment of syphilis.

### CROSS-REFERENCE(S) TO RELATED APPLICATION(S)

This application claims the benefit of U.S. Provisional Patent Application No. 63/402,787, filed Aug. 31, 2022; the content of which is hereby incorporated by reference in its entirety for all purposes.

### STATEMENT REGARDING SEQUENCE LISTING

The sequence listing XML associated with this application is provided in XML format and is hereby incorporated by reference into the specification. The name of the file XML containing the sequence listing is 3915-P1312WOUW_Sequence_Listing_ST26.xml. The XML file is 60,606 bytes; was created on Aug. 18, 2023; and is being submitted electronically via Patent Center with the filing of the specification.

### BACKGROUND

Syphilis is a sexually transmitted infection (STI) that can cause serious health problems without treatment. Infection develops in stages (primary, secondary, latent, and tertiary). Each stage can have different signs and symptoms.

Treatment for syphilis can be effective at targeting bacteria that are causative of syphilis (T. pallidum), however, treatment follows a positive clinical diagnosis based on laboratory testing that is time- and labor-intensive and at least somewhat inaccurate. For example, serologic assays are the current mainstay of laboratory diagnosis of syphilis, but involve confirmation by stepwise, algorithmic testing for treponemal and non-treponemal antibodies, increasing the turnaround time (TAT) of the test. Testing algorithms may be falsely negative in cases of early or incubating syphilis and, although serologic assays have rapid in-laboratory TAT, confirmation of results by overburdened Public Health Laboratories can take weeks to obtain a result. Direct microscopic methods to detect T. pallidum are at least as challenging as serologic assays and involve dedicated technical expertise; operational and staffing limitations mean virtually no clinical laboratories support darkfield or direct fluorescence microscopy for T. pallidum. Further, immunohistochemical stains in tissue are known to be cross-reactive with other spirochetes, complicating results.

The rapid and continued growth in syphilis cases in the United States, coupled with the time- and labor-intensive and error-prone laboratory testing procedures and limited operational and staffing support, means that there is not enough quality testing for syphilis. As a result, patients with syphilis are at greater risk for suffering with infection and without treatment for extended periods. This in turn is associated with serious deleterious health problems, including but not limited to significant maternal-fetal mortality and general health disparities, particularly for racial or ethnic minorities and men who have sex with men.

Accordingly, there is a need for improved compositions, kits, and methods for testing for syphilis that are configured and suitable for deployment and use in a patient point-of-care (POC) setting. The present disclosure addresses these and other long-felt but unmet needs in the field.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present disclosure provides new and improved laboratory testing approaches that improve speed and accuracy of laboratory diagnosis of syphilis. The disclosed elements are useful to disrupt the cycle of worsening infections by supporting prompt treatment decision-making. These rapid assays that target nucleic acids (e.g., molecular tests) of the causative organism, Treponema pallidum, meet the significant needs in the field and dramatically expand the limited testing options for syphilis.

In an aspect, the disclosure provides a site-specific primer comprising polynucleotide sequence configured to anneal to a T. pallidum nucleic acid for a loop-mediated isothermal amplification (LAMP) reaction.

In other aspects, the disclosure provides a composition for detection of a portion of a T. pallidum nucleic acid in a sample, the composition comprising a site-specific primer of the disclosure.

In embodiments, the composition comprises a plurality of site-specific primers comprising polynucleotide sequences configured to anneal to the T. pallidum nucleic acid in the sample, wherein the site-specific primer is of the plurality of site-specific primers.

In embodiments, the composition comprises a strand-displacing DNA polymerase configured to amplify the portion of the T. pallidum nucleic acid based on anneal positions of the plurality of site-specific primers to produce amplified products of the LAMP reaction; an intercalating agent, a probe, or a fluorophore for detection of the portion of the T. pallidum nucleic acid; or any combination thereof.

In embodiments, the plurality of site-specific primers comprises a forward inner primer (FIP), a backward inner primer (BIP), a forward outer primer (FOP), and a backward outer primer (BOP).

In embodiments, the plurality of site-specific primers further comprises a forward loop primer (FLP), a backward loop primer (BLP), or both.

In embodiments, the portion of the T. pallidum nucleic acid comprises at least a portion of a D1 domain of a 23S rRNA gene of the T. pallidum genome.

In embodiments, a sequence of the site-specific primer has at least 80% identity to a sequence selected from SEQ ID NOs: 1-12.

In embodiments, a sequence of the site-specific primer has at least 90% identity to a sequence selected from SEQ ID NOs: 1-12.

In embodiments, a sequence of the site-specific primer has 100% identity to a sequence selected from SEQ ID NOs: 1-12.

In embodiments, a forward inner primer (FIP) of the plurality of site-specific primers comprises SEQ ID NO: 3 or SEQ ID NO 9; a backward inner primer (BIP) of the plurality of site-specific primers comprises SEQ ID NO: 4 or SEQ ID NO: 10; a forward outer primer (FOP) of the plurality of site-specific primers comprises SEQ ID NO: 1 or SEQ ID NO: 7; a backward outer primer (BOP) of the plurality of site-specific primers comprises SEQ ID NO: 2 or SEQ ID NO: 8; a forward loop primer (FLP) of the plurality of site-specific primers comprises SEQ ID NO: 5 or SEQ ID NO: 11; and a backward loop primer (BLP) of the plurality of site-specific primers comprises SEQ ID NO: 6 or SEQ ID NO: 12.

In aspects, the disclosure provides a kit for detection of a portion of a T. pallidum nucleic acid in a sample, the kit comprising a site-specific primer or a composition of the disclosure.

In embodiments, the kit further comprises an instructional material for use of the kit in a method for detection of syphilis in the patient.

In an aspect, the disclosure provides a loop-mediated isothermal amplification (LAMP) reaction method for detection of a portion of a T. pallidum nucleic acid in a sample, the method comprising: contacting the sample with a site-specific primer or a composition of the disclosure and enabling the LAMP reaction to occur; and detecting the portion of the T. pallidum nucleic acid in amplified products of the LAMP reaction.

In embodiments, the detecting the portion of the T. pallidum nucleic acid in amplified products of the LAMP reaction comprises: contacting the sample with an intercalating agent, a probe, or a fluorophore for fluorescence detection of the portion of the T. pallidum nucleic acid; performing a lateral flow assay (LFA) for detection of the portion of the T. pallidum nucleic acid; performing agarose gel electrophoresis for detection of the portion of the T. pallidum nucleic acid; or any combination thereof.

In embodiments, the sample is a clinical sample and the method is a point-of-care (POC) method.

In embodiments, the sample comprises a swab sample, a cellular tissue specimen, or a body fluid.

In embodiments, the sample comprises the body fluid and the body fluid comprises whole blood, serum, plasma, peripheral blood mononuclear cells (PBMCs), a non-bloody body fluid, cerebrospinal fluid (CSF), or amniotic fluid.

In embodiments, the method has a limit of detection (LOD) of about 7-10 copies of the portion of the T. pallidum nucleic acid.

In embodiments, the method has specificity for detection of T. pallidum nucleic acids.

In embodiments, T. pallidum includes T. pallidum subspecies pertenue, T. pallidum subspecies endemicum, or T. pallidum subspecies pallidum.

In embodiments, T. pallidum includes T. pallidum subspecies pallidum and the method is for detection of syphilis in a patient.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1A shows an example plurality of site-specific primers for a loop-mediated isothermal amplification (LAMP) reaction, according to aspects of the disclosure.

FIG. 1B shows an example target nucleic acid, e.g., a T. pallidum nucleic acid (DNA), according to embodiments of the disclosure.

FIG. 1C shows an example annealing of a forward inner primer (FIP) to the target nucleic acid, according to embodiments of the disclosure.

FIG. 1D shows an example result from DNA polymerase extension from the FIP, according to embodiments of the disclosure.

FIG. 1E shows an example annealing of a forward outer primer (FOP) to the target nucleic acid, displacing the FIP/extension strand therefrom, according to embodiments of the disclosure.

FIG. 1F shows an example result from DNA polymerase extension from the FOP, according to embodiments of the disclosure.

FIG. 1G shows an example annealing of a backward inner primer (BIP) to the target nucleic acid, according to embodiments of the disclosure.

FIG. 1H shows an example result from DNA polymerase extension from the BIP, according to embodiments of the disclosure.

FIG. 1I shows an example annealing of a backward outer primer (BOP) to the target nucleic acid, according to embodiments of the disclosure.

FIG. 1J shows an example result from DNA polymerase extension from the BOP, as well as an annealing of a FIP to the BIP/extension strand product (from FIG. 1H) and subsequent polymerase extension (arrow), according to embodiments of the disclosure.

FIG. 1K shows example results from initial steps of the LAMP reaction, wherein complementary portions of the resultant nucleic acids hybridize to form dumbbell-shaped nucleic acids that include loop portions, according to embodiments of the disclosure.

FIG. 1L shows an example annealing of a forward loop primer (FLP; loop forward (LF)) and a backward loop primer (BLP; loop backward (LB)), as well as annealing of a FIP and a BIP, to the dumbbell-shaped nucleic acids, according to embodiments of the disclosure.

FIG. 1M shows an example result from amplification steps of the LAMP reaction, wherein amplification products having various secondary structures are formed and provide the basis for detection, according to embodiments of the disclosure.

FIG. 2A shows example representative melt curves comparing true positives and off-target amplifications demonstrating that melt curves from non-specific amplification are readily distinguishable from true positives; NTC peak detected during amplification from residual clinical specimens (BLUE) was right-shifted relative to positive controls (POS CTRL, RED, PINK), according to embodiments of the disclosure.

FIG. 2B shows example representative melt curves comparing true positives and off-target amplifications demonstrating that melt curves from non-specific amplification are readily distinguishable from true positives; QuantStudio™ software call of a peak at 80.5° C. for Case 44, replicate 2 (black arrow) determined as negative by manual review, according to embodiments of the disclosure.

FIG. 2C shows example representative melt curves comparing true positives and off-target amplifications demonstrating that melt curves from non-specific amplification are readily distinguishable from true positives; QuantStudio™ software call of a peak at 79.9° C. for Case 55, both replicates (black arrow) determined as negative by manual review, according to embodiments of the disclosure.

FIG. 2D shows example representative melt curves comparing true positives and off-target amplifications demonstrating that melt curves from non-specific amplification are readily distinguishable from true positives, according to embodiments of the disclosure; 11 off-target amplifications were detected in an initial experiment with gBlock specificity controls and were distinguishable from positive controls and T. pallidum gDNA templates. Five of the eleven amplifications had C_{T}>40.

FIG. 3A shows an example multiple sequence alignment from sequencing results for positive cases and controls, according to embodiments of the disclosure; alignment of trimmed sequences from positive validation cases (#36-40, #48), gBlock control, synthetic positive control reactions, and reference wild type and synthetic sequences demonstrated 100% identity between true positives and wild type gBlock control with reference strain NR_076156. Sequenced synthetic controls demonstrated the expected, introduced polymorphisms and were readily distinguishable from wild type sequences. The more dilute LAMP product (level 3) produced a longer usable sequence. Polymorphic motifs 1 and 2 are indicated with single or double asterisks above the consensus sequence, top.

FIG. 3B shows an example multiple sequence alignment from sequencing results for positive cases and controls, according to embodiments of the disclosure; representative sequence results highlight synthetic polymorphic motif 1 reliably seen in the sequenced products and complementary to wild type sequence.

FIG. 3C shows an example multiple sequence alignment from sequencing results for positive cases and controls, according to embodiments of the disclosure; representative sequence results highlight synthetic polymorphic motif 2 reliably seen in the sequenced products and complementary to wild type sequence.

FIG. 4 shows an example multisequence alignment from Basic Local Alignment Search Tool (BLAST) results with a 125nt product representing workable sequence from sequenced LAMP products, according to embodiments of the disclosure. The figure demonstrates that there are consistent polymorphisms that distinguish recovered T. pallidum sequence (top line, indicated as Query_500765) from other, closely related organisms (e.g., other, non-pallidum treponemes). Notably, the rabbit pathogen T. paraluiscuniculi has a 100% nucleotide identity to T. pallidum at rRNA and other loci but is not a human pathogen.

FIG. 5 shows an example representative sequencing of a LAMP product with Tₘ values not matching control, according to embodiments of the disclosure; the sequence did not contain T. pallidum sequence.

FIG. 6A shows example LAMP amplification curves of genomic DNA from NICHOLS T. pallidum strain, shown for example LAMP primer sets P3L2 and PIL1, according to embodiments of the disclosure.

FIG. 6B shows example LAMP amplification curves of genomic DNA from SS14 T. pallidum strain, shown for example LAMP primer sets P3L2 and PIL1, according to embodiments of the disclosure.

FIG. 6C shows example LAMP amplification curves of genomic DNA from UW091B T. pallidum strain, shown for example LAMP primer sets P3L2 and PIL1, according to embodiments of the disclosure.

FIG. 6D shows example LAMP amplification curves of genomic DNA from UW228B T. pallidum strain, shown for example LAMP primer sets P3L2 and PIL1, according to embodiments of the disclosure.

FIG. 7 shows example aligned sequences from LAMP-amplified nucleic acids extracted from cultured strains of T. pallidum, demonstrating 100% identity over the expected region (large consensus bars) and the expected divergence from the synthetic positive control at the two polymorphic residues (small consensus bars), according to embodiments of the disclosure.

FIG. 8A shows primers from set P3L2 mapped to an example target 23S rRNA locus of T. pallidum, according to embodiments of the disclosure.

FIG. 8B shows primers from set PIL1 mapped to an example target 23S rRNA locus of T. pallidum, according to embodiments of the disclosure.

FIG. 9 shows a flowchart for an example loop-mediated isothermal amplification (LAMP) reaction method for detection of a portion of a T. pallidum nucleic acid in a sample, according to embodiments of the disclosure; anneal positions refer to positions where primers anneal or base-pair with the target nucleic acid.

### DETAILED DESCRIPTION

This disclosure provides isothermal loop-mediated amplification (LAMP) assays that detect T. pallidum nucleic acids with high sensitivity and specificity, for use in clinical environments at patient point-of-care (POC). The example assays of the disclosure have been validated for clinical use under the CLIA-88 framework, and have a limit of detection of about 7-10 genomes per reaction; 100% accuracy in residual clinical specimens known to contain T. pallidum DNA; and 100% specificity in a panel of >100 species of bacteria, including closely related Treponema spp., and human DNA. The example assays can be used for testing of direct patient specimens, including but not necessarily limited to swab samples, cellular tissue specimens, body fluids, and blood.

The disclosure provides LAMP reagents and assays useful for testing for presence of T. pallidum in samples obtained from human or animal subjects or specimens, for example. Because the methods are isothermal and involve relatively minimal laboratory equipment and expertise for use, they can be deployed for use within geographical areas or individual organizations that suffer from understaffing, underfunding, or other problems that are currently preventing accessible and effective care for syphilis patients. As a result, the disclosed site-specific primers, compositions, kits, methods, and other features of the disclosure provide an effective solution for syphilis testing that overcomes issues present in many modern healthcare settings.

### Site-Specific Primers

In various aspects, the disclosure provides one or more site-specific primers that comprise one or more polynucleotide sequences that share at least a certain amount of percent identity with a T. pallidum nucleic acid, such that the polynucleotide sequences are configured to base pair with or anneal to the T. pallidum nucleic acid for carrying out a loop-mediated isothermal amplification (LAMP) reaction for detection of the T. pallidum nucleic acid in a sample. A plurality of site-specific primers of the disclosure comprises a plurality of polynucleotide sequences that are configured to base pair with or anneal to the T. pallidum nucleic acid in the sample for the LAMP reaction. While one target nucleic acid for T. pallidum is targeted for LAMP detection as an example in this disclosure, additional or other or multiple target nucleic acids, e.g., of T. pallidum, can be implemented.

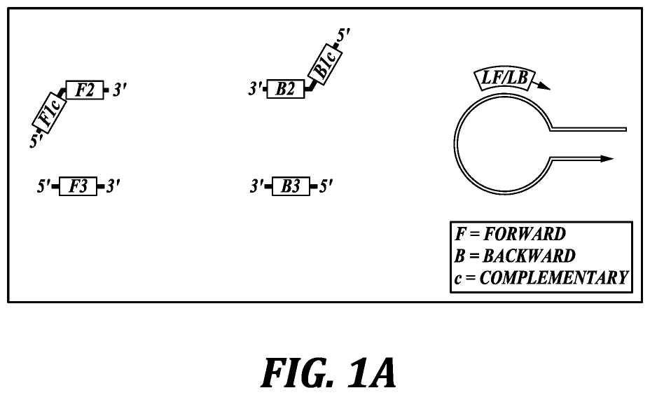

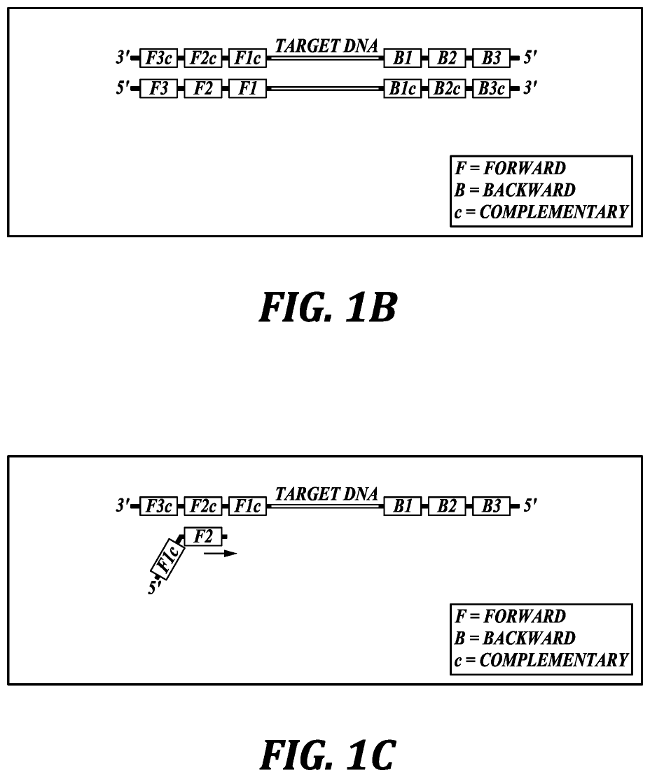

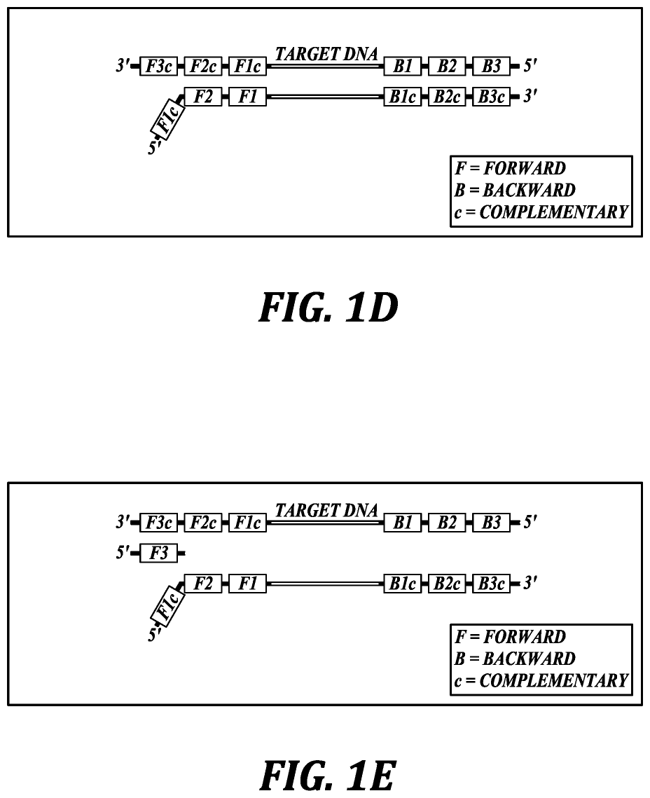

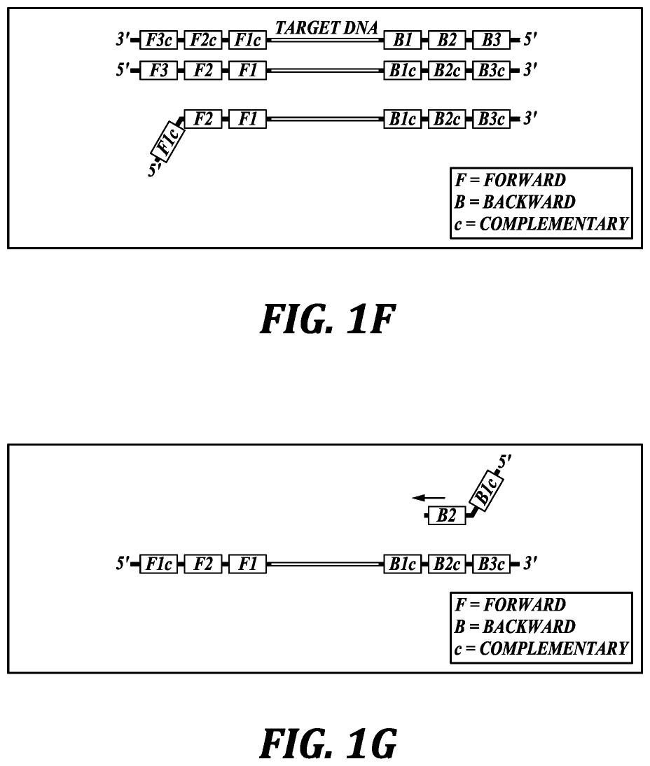

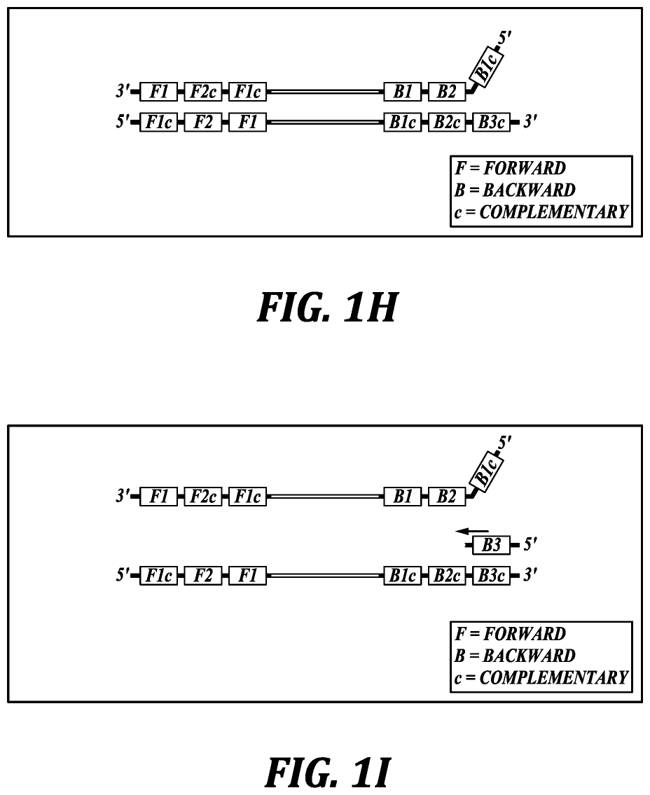

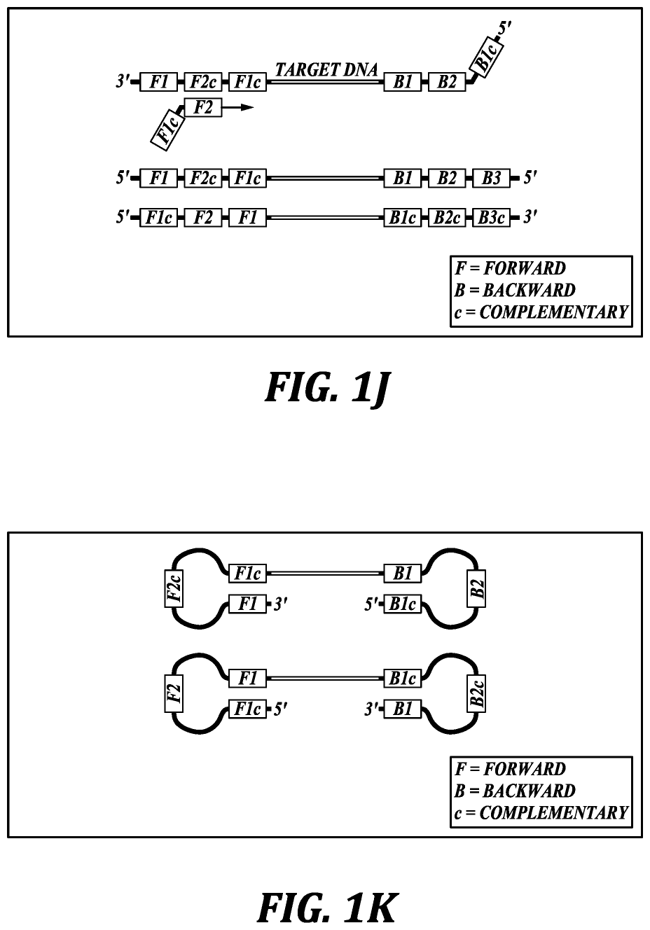

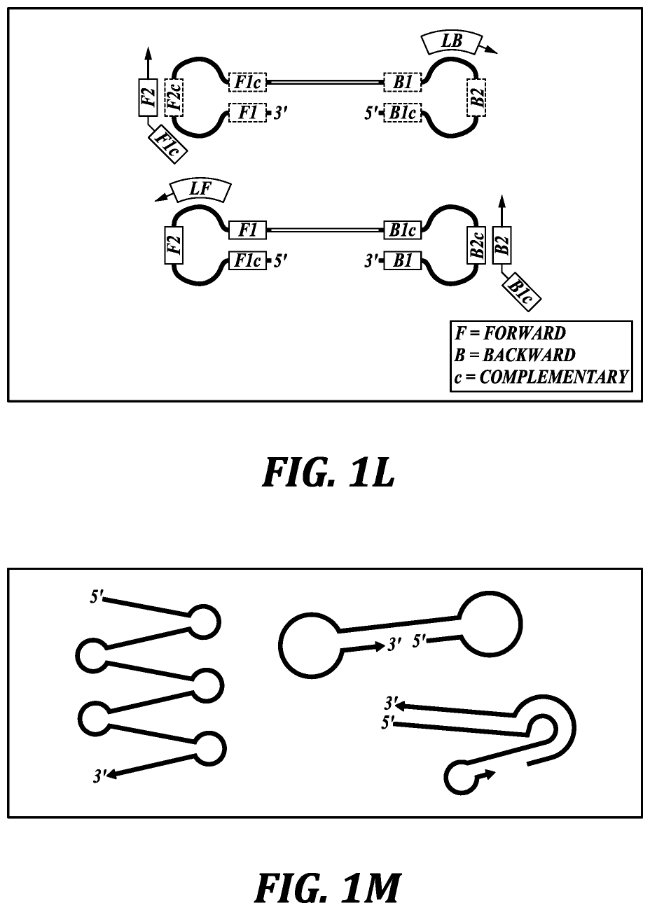

As shown at FIG. 1A, an example plurality of site-specific primers for a loop-mediated isothermal amplification (LAMP) reaction, according to aspects of the disclosure, includes a forward inner primer (FIP; designated ‘F1c-F2’), a backward inner primer (BIP; designated ‘B2-B1c), a forward outer primer (FOP; designated ‘F3’), and a backward outer primer (BOP; designated ‘B3’). While the FIP, BIP, FOP, and BOP can be considered to be a first plurality of primers, in at least some embodiments, the plurality of site-specific primers further comprises a forward loop primer (FLP; designated LF), a backward loop primer (BLP; designated LB), or both for a second plurality of primers. While the LAMP reaction occurs with the first set of primers, one or more of the loop primers (FLP, BLP) enhances the speed or processivity of amplification, when present in the reaction, by annealing to loop portions of nucleic acids and providing binding sites for a strand-displacing DNA polymerase to for effectively produce amplified products of the LAMP reaction. The LAMP reaction can therefore proceed in the absence of any loop primer, in the presence of one loop primer (i.e., one of FLP, BLP), or in the presence of both loop primers (i.e., both of FLP, BLP), with enhanced speed or processivity of amplification with more loop primers included. As such, in embodiments, a strand-displacing DNA polymerase is included and implemented to amplify the portion of the T. pallidum nucleic acid, based on anneal positions of the plurality of site-specific primers, to produce amplified products of the LAMP reaction as explained in more detail herein.

The LAMP reaction proceeds in two stages that includes 1) nucleic acid amplification and 2) amplified product detection. A first-stage slow rate amplification reaction is followed by (or co-occurs with) a plurality of second-stage fast rate amplification reactions that can be simultaneously monitored in real-time, such that a rapid rate of amplification is indicative of the presence of a site of interest of a target nucleic acid (e.g., real-time detection with intercalating dyes or probes). As an alternative to real-time detection, products of the amplification can be detected or analyzed after the amplification occurs, for example, with LFA, DNA sequencing, or a gel electrophoresis.

Example steps of a LAMP reaction are shown at FIGS. 1B-1M. FIG. 1B shows an example target nucleic acid, e.g., a T. pallidum nucleic acid (designated ‘TARGET DNA’). While in example embodiments the portion of the T. pallidum nucleic acid comprises at least a portion of a D1 domain of a 23S rRNA gene of the T. pallidum genome, other portions of the T. pallidum genome or transcriptome can be used for the LAMP reaction without departing from the scope of the disclosure. In the shown example, the target DNA is double stranded and contains sequences (F1, F2, F3; B1, B2, B3) base-paired with corresponding complementary sequences (F1c, F2c, F3c; B1c, B2c, B3c).

With respect to steps of the LAMP reaction, a forward inner primer (FIP) is annealed to the top strand of the target nucleic acid with base-pairing between complementary regions of the FIP and the target DNA, in this case, F2 and F2c, respectively (FIG. 1C). The FIP contains a 5′ overhang sequence portion, shown as F1c, that is not complementary to the adjacent target DNA sequence (F3c) and is at least mostly the same as a corresponding sequence of the target DNA (F1c). Extension from the 3′ end of the FIP occurs with a strand-displacing DNA polymerase to produce an extended complementary strand that base pairs with the upper target DNA strand but also retains the 5′ overhang, as shown at FIG. 1D.

While any suitable strand-displacing DNA polymerase can be used in the LAMP assay, in at least some embodiments, Bst polymerase is used. Bst is useful for detecting and amplifying both DNA and RNA templates (i.e., Bst can perform both LAMP and reverse-transcription LAMP or RT-LAMP reactions, without a separate reverse transcription step with a reverse transcriptase). In this manner, DNA, RNA, or both DNA and RNA target nucleic acids can be detected and amplified by the assay. Other enzymes can be used for LAMP, but the assay generally includes use of a strand-displacing polymerase. If Bst is not used and a dedicated DNA polymerase is selected, then addition of reverse transcriptase can be included if RNA templates are to be amplified. In instances where Bst is utilized, an example source of Bst enzyme for use in the assay is New England Biolabs® WarmStart® LAMP reagents, however, Bst from other sources, or other derivatives of Bst, can also be used in the LAMP assay.

Referring now to FIG. 1E, a forward outer primer (FOP; shown as F3) is annealed to the top strand of the target nucleic acid, thereby displacing the FIP/extension strand therefrom, when present. Extension from the FOP with the strand-displacing DNA polymerase occurs to form a complementary target DNA lower strand which creates additional templates for BIP and BOP extensions and allows the FIP/extension strand to be stably displaced from the target DNA, when present (as shown at FIG. 1F). The FIP/extension strand, i.e., the lower strand shown at FIG. 1F as not base-paired with other sequences, contains target DNA sequence positioned between the left (F1c, F2, F1) and right (B1c, B2c, B3c) sequence portions. Since both the FOP and FIP primers are simultaneously present within the LAMP reaction, both FOP and FIP polymerase extensions occur in parallel within the LAMP reaction, and FOP extension events provide more copies of the template (e.g., target DNA) for use by the FIP, enabling exponential amplification and resultant detection.

The FIP/extension strand (FIG. 1G) is used as the template for strand-displacing DNA polymerase extension from a BIP (shown as B2-B1c) that anneals to the corresponding (right-hand side) portion of the strand (i.e., B2 anneals with B2c). This extension forms a BIP/extension strand (i.e., top strand of FIG. 1H, that is, the strand containing F1-F2c-F1c-TARGET-B1-B2-B1c). The BIP contains a 5′ overhang sequence portion, shown as B1c, that is not complementary to the adjacent sequence (B3c) and is at least mostly the same as a corresponding sequence of the target DNA (B1c).

A backward outer primer (BOP; shown as B3 at FIG. 1I) anneals to the FIP/extension strand, thereby displacing the BIP/extension strand therefrom, when present. Extension from the BOP with the strand-displacing DNA polymerase occurs to form a complementary upper strand and allows the BIP/extension strand to be stably displaced (FIG. 1J). Meanwhile, a FIP anneals to the BIP/extension strand (FIG. 1J, top) and extension occurs therefrom to form a complementary lower strand containing 5′-F1c-F2-F1-TARGET-B1c-B2c-B1-3′ (see FIG. 1K).

The initial products form self-loops, such that F1 base pairs with F1c and B1 base pairs with B1c, as shown at FIG. 1K. This dumbbell structure, and other similar “loop” structures generated by the reaction, form thermodynamically stable templates with exposed single-stranded regions (e.g., F2c, B2 of FIG. 1L) that are readily available for base pairing with one or more loop primers. Base pairing of loop primers, e.g., as shown at FIG. 1L, occurs favorably (LF, loop forward primer (also referred to as ‘FLP’); LB, loop backward primer (also referred to as ‘BLP’)) and additional strand-displacing DNA polymerase extension occurs, from the base-paired loop primers and any other base-paired primers in the reaction mixture (e.g., FIP and BIP, as shown at FIG. 1L), to form a myriad of amplified products of the LAMP reaction, as shown at FIG. 1M. These amplified products are detectible with any of a variety of DNA detection techniques, including but not necessarily limited to use of an intercalating agent, a probe, or a fluorophore, or any combination thereof, for example.

Particular sequences of primers selected for use in the LAMP assays of the disclosure depends, among other things, on which portion of the T. pallidum genome or transcriptome is to be detected in the assay. This disclosure uses, as an example, primers comprising sequences configured to anneal to and enable amplification of a portion of the 23S rRNA gene with the LAMP reaction, however, other T. pallidum nucleic acids or other portions of the 23S rRNA gene can be amplified, in embodiments, for detection of T. pallidum.

In embodiments, a sequence of a site-specific primer used in a LAMP reaction has at least about 50%, at least about 55%, at least about 60%, at least about 65%, at least about 70%, at least about 75%, at least about 80%, at least about 85%, at least about 90%, at least about 95%, or at least about 100% or 100% identity to a sequence selected from SEQ ID NOs: 1-12. In embodiments, sequences of multiple primers used in the LAMP reaction have at least about 50%, at least about 55%, at least about 60%, at least about 65%, at least about 70%, at least about 75%, at least about 80%, at least about 85%, at least about 90%, at least about 95%, or at least about 100% or 100% identity to sequences selected from SEQ ID NOs: 1-12.

In embodiments, a plurality of site-specific primers (e.g., P3L2 set of primers) includes 6 primers having sequences at least about 50%, at least about 55%, at least about 60%, at least about 65%, at least about 70%, at least about 75%, at least about 80%, at least about 85%, at least about 90%, at least about 95%, or at least about 100% or 100% identity to a sequence selected from SEQ ID NOs: 1-6.

In embodiments, a plurality of site-specific primers (e.g., PIL1 set of primers) includes 6 primers having sequences at least about 50%, at least about 55%, at least about 60%, at least about 65%, at least about 70%, at least about 75%, at least about 80%, at least about 85%, at least about 90%, at least about 95%, or at least about 100% or 100% identity to a sequence selected from SEQ ID NOs: 7-12.

In embodiments, a forward inner primer (FIP) of the plurality of site-specific primers comprises SEQ ID NO: 3 or SEQ ID NO 9; a backward inner primer (BIP) of the plurality of site-specific primers comprises SEQ ID NO: 4 or SEQ ID NO: 10; a forward outer primer (FOP) of the plurality of site-specific primers comprises SEQ ID NO: 1 or SEQ ID NO: 7; a backward outer primer (BOP) of the plurality of site-specific primers comprises SEQ ID NO: 2 or SEQ ID NO: 8; a forward loop primer (FLP) of the plurality of site-specific primers comprises SEQ ID NO: 5 or SEQ ID NO: 11; and a backward loop primer (BLP) of the plurality of site-specific primers comprises SEQ ID NO: 6 or SEQ ID NO: 12.

### Compositions

In various aspects, the disclosure provides a composition for detection of a portion of a T. pallidum nucleic acid in a sample, the composition comprising a site-specific primer or a plurality of site-specific primers of the disclosure. The composition can comprise one, two, three, four, five, six, or more site-specific primers in any suitable form, including but not limited to lyophilized, solubilized, and the like. For example, a composition can include a site-specific primer that is lyophilized for storage or transport. When the primer is to be used for a LAMP assay, it can be solubilized, for example by adding water or solvent, and the solubilized primer can be added to a reaction mixture comprising a target nucleic acid. Alternatively, the composition can comprise a plurality of site-specific primers, for example, in a lyophilized form, such that by adding water or solvent to the lyophilized plurality of site-specific primers, the plurality of site-specific primers is solubilized and ready for use in the LAMP assay by being combined with the target nucleic acid.

In embodiments, a composition comprises one or more site-specific primers, optionally in combination with a strand-displacing DNA polymerase. The one or more site-specific primers and the strand-displacing DNA polymerase can be lyophilized for storage or transport and solubilized when ready for use.

In embodiments, a composition comprises one or more site-specific primers, optionally in combination with a detection reagent for detection of amplification products of the LAMP reaction, optionally in real time. The detection reagent can comprise an intercalating agent, a probe, or a fluorophore for fluorescence detection of the portion of the T. pallidum nucleic acid, e.g., as copy number of the portion of the T. pallidum nucleic acid increases as the LAMP reaction of a positive sample proceeds.

In embodiments, a composition comprises one or more site specific primers, a strand-displacing DNA polymerase, and a detection reagent together in combination. For example, the composition can include reagents for amplification and detection by the LAMP assay, except for a sample that possibly contains a target nucleic acid. The combination composition can be provided as a lyophilized powder of the multiple reagents together, which can be solubilized by addition of water or solvent to produce a reaction tube sans sample. The contents of the reaction tube (e.g., ‘master mix’) can be contacted with the sample to enable the LAMP reaction.

In embodiments, elements of the LAMP reaction (e.g., individual primers of the plurality of site-specific primers, strand-displacing DNA polymerase, detection agent(s)) are included within separate, individual, compartmentalized compositions that are combinable for enabling a LAMP assay.

### Kits

In certain aspects, the disclosure provides a kit for detection of a portion of a T. pallidum nucleic acid in a sample, the kit comprising a site-specific primer or a composition of the disclosure. In embodiments, the kit further comprises an instructional material for use of the kit in a method for detection of syphilis in the patient. The instructional material can include print or graphic materials containing instructions for use by a testing facility or care center, for example. These materials can optionally be in combination with additional resources for the kit, for example, a web address that directs a web browser of a computational device (e.g., smartphone, tablet, computer) to a website for further information such as literature, videos, and the like.

In embodiments, kits of the disclosure can be generated at a point of care (POC) or at a service provider facility using individually sourced elements of the kit, or can be obtained commercially for clinical testing purposes.

### Methods

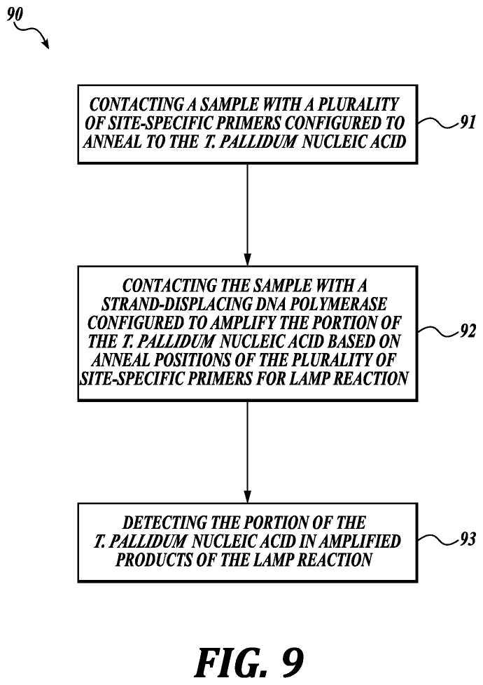

In various aspects, the disclosure provides a loop-mediated isothermal amplification (LAMP) reaction method for detection of a portion of a T. pallidum nucleic acid in a sample. In embodiments, the sample contains nucleic acids released from cells or organisms; for example, sample preparation steps can occur to release nucleic acids, including T. pallidum nucleic acid when present, into the solution of the sample for operability of primers and the LAMP reactions of the disclosure. As shown at FIG. 9, such a method 90 can comprise, at step 91, contacting a sample with a plurality of site-specific primers configured to anneal to the T. pallidum nucleic acid; at step 92, contacting the sample with a strand-displacing DNA polymerase configured to amplify the portion of the T. pallidum nucleic acid based on anneal positions of the plurality of site-specific primers for a LAMP reaction; and at step 93, detecting the portion of the T. pallidum nucleic acid in amplified products of the LAMP reaction.

In embodiments, the detecting the portion of the T. pallidum nucleic acid in amplified products of the LAMP reaction comprises: contacting the sample with an intercalating agent, a probe, or a fluorophore for fluorescence detection of the portion of the T. pallidum nucleic acid; performing a LFA for detection of the portion of the T. pallidum nucleic acid; performing agarose gel electrophoresis for detection of the portion of the T. pallidum nucleic acid; or any combination thereof.

In embodiments, the sample is a clinical sample, and the method is a point-of-care (POC) method. In embodiments, the sample comprises a swab, a cellular tissue specimen, or a body fluid. In embodiments, the sample comprises the body fluid and the body fluid comprises whole blood, serum, plasma, peripheral blood mononuclear cells (PBMCs), a non-bloody body fluid, cerebrospinal fluid (CSF), or amniotic fluid.

In embodiments, the method has a limit of detection (LOD) of about 7-10 copies of the portion of the T. pallidum nucleic acid. In embodiments, the method has specificity for detection of T. pallidum nucleic acids.

In embodiments, T. pallidum includes T. pallidum subspecies pertenue, T. pallidum subspecies endemicum, or T. pallidum subspecies pallidum, but in particular embodiments, T. pallidum includes T. pallidum subspecies pallidum and the method is for detection of syphilis in a patient.

After detection of a presence or absence of syphilis in a sample from a patient, a healthcare provider can suggest, recommend, update, implement, or alter a course of care or treatment for the patient based on the test result. In at least some embodiments wherein the test result is positive for syphilis, the method further comprises prescribing a course of treatment for the patient. The course of treatment can include, for example, treatment of the patient with an antibiotic (e.g., penicillin, tetracycline, doxycycline, etc.) or another therapy to treat syphilis.

### EXAMPLES

### Example 1

Treponema pallidum Detection by Loop-Mediated Isothermal Amplification Validation

This example describes the development and validation of a loop-mediated amplification (LAMP) assay that detects Treponema pallidum (T. pallidum, TP) nucleic acids with high-sensitivity and specificity for clinical use under the CLIA-88 framework. This molecular assay can be used for testing of direct patient specimens, including swabs, cellular tissue specimens, and body fluids other than blood, serum, or plasma. Specifically, this assay detects T. pallidum, the etiologic agent that causes syphilis, in clinical specimens by targeting and amplifying the 23S rRNA gene using the loop-mediated amplification (LAMP) reaction. The performance of this assay was tested using 1) extracted genomic DNA (gDNA) from cultured T. pallidum clinical and laboratory isolates; 2) a variety of residual clinical specimens with closely related treponemes and more distantly related, clinically relevant organisms, and human DNA; and 3) synthetic dsDNA controls representing the homologous 23S rRNA sequences of closely related treponemes and uncultivable and/or uncommon organisms found in anatomically relevant sites. As described in detail below, a systematic evaluation of the performance of this assay has demonstrated this assay to be a novel adjunct in syphilis diagnosis that can speed turnaround time and resolve ambiguous results from stepwise serologic assays, the current mainstay of syphilis diagnosis.

### A. Methods

### A1. Primer Design

The D1 domain of the 23S rRNA gene of the TP genome was selected to maximize the likelihood of sequence divergence from closely related treponemes with NR_076156 (TP strain Nichols) selected as reference. Two candidate sets of six LAMP primers were designed against the proximal 546 nt using the New England Biolabs LAMP primer design tool <https://lamp.neb.com/#!/>. Two candidate primer sets (Integrated DNA Technologies) were selected based on bioinformatic analysis (Table 1, selected primer set only) and based on their performance in preliminary experiments assessing sigmoidal increase in fluorescence and cycle threshold (C_{T}) value using gDNA extracted from cultures of TP Nichols and two clinical isolates as template (see Example 3). A single primer set was selected for validation (Table 1).

A2. In silico Analysis of 23S rRNA Sequences from Closely and Distantly Related Organisms

The 23S target and primer binding sites were analyzed by BLAST using the NCBI nr/nt database in multiple iterations: 1) within genus Treponema excluding Treponema pallidum; 2) excluding genus Treponema; and 3) within each subspecies of T. pallidum. The 23S target was 100% identical to all subspecies of T. pallidum (TP subsp pertenue, TP subsp endemicum, and TP subsp pallidum) and to the rabbit-infecting species T. paraluiscuniculi. Maximum homology for each primer to non-pallidum treponemes ranged from 100% (F3; B2 portion of BIP) to 59.1% (F1c portion of FIP) and the species of maximum homology varied across primers (Table 1).

TABLE 1
Primer Sequences and Maximum Sequence Homology to Non-Pallidum
Treponemes*

| | Non- | | |
|---|---|---|---|
| | T. pallidum | | |
| | Homology | | Species with |
| | (SNPs/gaps; % | | Max |
| Primer | identity) | Primer Sequence | Homology |
| F3 | 0 (100%) | 5′-AAAAGCCTCGGGGAGGAG-3′ | T. phagedenis |
| | | SEQ ID NO: 1) | |
| B3 | 6 (70.0%) | 5′-AGACACAGGTTTAGGCTCCT- | T. ruminis |
| | | 3′ (SEQ ID NO: 2) | |
| FIP | — | 5′- | |
| | | TCAGGAAGGCAATGACAAGGCT | |
| | | TCCTGTGATCCGGGGATG-3′ | |
| | | (SEQ ID NO: 3) | |
| FIP (F2) | 3 (83.3%) | 5′-TCCTGTGATCCGGGGATG-3′ | T. |
| | | (SEQ ID NO: 57) | azonutricium |
| | | | & |
| | | | T. vincentii |
| FIP (F1c) | 9 (59.1%) | 5′. | T. azonutricium |
| | | AGCCTTGTCATTGCCTTCCTGA- | |
| | | 3′ (SEQ ID NO: 58) | |
| BIP | — | 5′- | |
| | | AAGGCGAAACTGGGTGAACTGA | |
| | | TTCGCTCG CCACTACTTTC-3′ | |
| | | (SEQ ID NO: 4) | |
| BIP (B2) | 1 (95.5%) | 5′- | T. phagedenis, |
| | | AAGGCGAAACTGGGTGAACTGA-phagedenis | |
| | | 3′ (SEQ ID NO: 59) | |
| BIP (B1c) | 0 (100%) | 5′-GAAAGTAGTGGCGAGCGAA-3′T. | |
| | | (SEQ ID NO: 60) | T. vincentii, |
| | | | T. medium, |
| | | | & |
| | | | Treponema |
| | | | sp. |
| Loop Fwd | 1 (95.0%) | 5′-CTGTCGGGTTACCCCATTCG-3′T. | T. parvum |
| | | (SEQ ID NO: 5) | |
| Loop Back | 1 (96.0%) | 5′- | T. peruense, |
| | | GGGAAAAGAAATCAAGAGAGA | T. rectale, & |
| | | TTCC-3′ (SEQ ID NO: 6) | Treponema |
| | | | sp. |
| TPAL_Seq_Fw | N/A | 5′-TCCTGTGATCCGGGGATG-3′ | N/A |
| | | (SEQ ID NO: 61) | |
| TPAL_Seq Rv | N/A | 5′-TTCGCTCGCCACTACTTTC-3′ | N/A |
| | | (SEQ ID NO: 62) | |

*Excludes T. paraluiscuniculi, a rabbit pathogen, with 100% identity to T. pallidum at multiple loci; see Table 4 for more information.

### A3. Reaction Conditions

Reagents were sourced from New England Biolabs as the WarmStart® fluorescent LAMP/RT-LAMP Kit (2× Master Mix with UDG; Cat #E1708). Reaction mixtures were prepared as described in Table 2 and were incubated at 65° C. for 40 min on a QuantStudio 5 with fluorescence assessed at 1 min intervals. A melt curve analysis was performed at the end of the reaction.

TABLE 2
Reaction Conditions

| | Volume per |
|---|---|
| | 25 mcL Reaction |
| Component | (mcL) |
| 2X WarmStart LAMP | 12.5 |
| Mix | |
| 10X primer set (with | 2.5 |
| tRNA) | |
| s50X fluorescent dye | 0.5 |
| water | 4.5 |
| template | 5 |
| total volume | 25 |

The 10× primer mixture was prepared from 100 mcM primer stock solutions diluted to 10× working stock concentrations of: 2 mcM (F3 and B3); 4 mcM (Loop Fwd, Loop Back); and 16 mcM (FIP and BIP). Carrier tRNA (from Baker's Yeast) was included in the primer mix at 1 mg/mL.

### A4. Strains and Clinical Specimens

The cultured strains and residual clinical DNA eluates included in the analysis are listed in Table 3.

TABLE 3
List of Included Cultured Strains and Clinical Specimens with
Specimen Description and Major Findings from Clinical Testing.

| Study | | | | Clinical Testing |
|---|---|---|---|---|
| ID | MDXID | EXID | Specimen Type | Results |
| E01 | UW99B | n/a | Cultured clinical | n/a |
| | | | isolate; | |
| | | | EasyMag | |
| | | | Extraction | |
| | | | (MDx) | |
| E02 | UW99B | n/a | Cultured clinical | n/a |
| | | | isolate; | |
| | | | EasyMag | |
| | | | Extraction | |
| | | | (MDx) | |
| E05 | UW228B | n/a | Cultured clinical | n/a |
| | | | isolate; | |
| | | | EasyMag | |
| | | | Extraction | |
| | | | (MDx) | |
| E09 | TP Nichols | n/a | Cultured TP | n/a |
| | | | Nichols; | |
| | | | EasyMag | |
| | | | Extraction | |
| | | | (MDx) | |

Clinical Specimens Containing Non-pallidum Treponema specificity controls

| | | | | |
|---|---|---|---|---|
| 1 | 220404_060 | E220404060 | Bronchoalveolar | [Major abundance]: |
| | | | Lavage | Bacteria of the |
| | | | | Family |
| | | | | Treponemataceae |
| 2 | 220428_040 | E220429027 | Pleural Tissue | MULTE |
| 3 | 220307_019 | E220307014 | Bronchoalveolar | [Moderate |
| | | | Lavage Lung, | abundance]: |
| | | | Right | Bacteria of the |
| | | | | Family |
| | | | | Treponemataceae |
| 4 | 220224_102 | E220225057 | PET Lung Right | NGS: Bacteria of the |
| | | | Biopsy Core | Family |
| | | | | Treponemataceae |
| | | | | (T. pallidum Assay) |
| 5 | 220416_021 | E220417012 | Bronchoalveolar | [Moderate |
| | | | Lavage | abundance]: |
| | | | | Treponema species |
| 6 | 220420_068 | E220421088 | Paraffin | [Major abundance]: |
| | | | embedded | Treponema species |
| | | | tissue Oral | |
| | | | Cavity | |
| 7 | 220326_029 | E220327008 | Oral Supra and | [Major abundance]: |
| | | | Subgingival | Treponema species |
| | | | Biofilm B Sub R | |
| | | | eSwab | |
| 8 | 210823_13 | E210823015 | Bronchoalveolar | Multiple bacterial |
| | | | Lavage Lung | DNA templates |
| | | | | (non-pallidum case) |
| 9 | 201119_87 | E201119079 | Fluid Abscess | [Minor abundance]: |
| | | | Fluid | Streptococcus |
| | | | | constellatus, |
| | | | | Fusobacterium |
| | | | | nucleatum, |
| | | | | Treponema sp. |
| 10 | 210922_068 | | Not found; | |
| | | | omitted | |
| 11 | 220529_041 | E220531032 | Right Mandible | [Minor abundance]: |
| | | | | Treponema |
| | | | | socranskii |
| 12 | 210821_68 | E210822072 | Tissue Paraffin | Multiple bacterial |
| | | | embedded | DNA templates |
| | | | tissue Mandible | (non-pallidum case) |
| | | | Righ | |
| 13 | 211005_021 | | Not found; | |
| | | | omitted | |
| 14 | 210609_81 | E210609083 | Tissue, Paraffin | Multiple bacterial |
| | | | embedded Lip, | DNA templates |
| | | | Lower Left | (non-pallidum case) |
| 15 | 211201_084 | E211201087 | Tissue Liver | Treponema |
| | | | GROUND IN | lecithinolyticum |
| | | | SALINE | (NGS) |
| 16 | 210818_49 | E210818052 | Paraffin | Multiple bacterial |
| | | | embedded | DNA templates |
| | | | tissue BAL, | (non-pallidum case) |
| | | | Right Middle | |
| | | | Lobe | |
| 17 | 220418_030 | E220420096 | Paraffin | [Trace abundance]: |
| | | | embedded | Treponema |
| | | | tissue Right | denticola |
| | | | Epiglottis | |
| | | | Lingual surface | |
| | | | Larynx | |
| 18 | 201107_31 | E201108030 | Tissue Ear, Left | [Moderate |
| | | | MASTOID | abundance]: |
| | | | CONTENT | Porphyromonas |
| | | | | gingivalis, |
| | | | | Treponema |
| | | | | denticola |
| 19 | 200320_6 | E200320006 | Paraffin | Treponema |
| | | | embedded | denticola |
| | | | tissue Tonsil | |

Clinical Specimens Serving as Non-Treponema specificity controls

| | | | | |
|---|---|---|---|---|
| 20 | 210224_72 | E210224070 | Paraffin | Brachyspira aalborgi |
| | | | embedded | |
| | | | tissue | |
| | | | TRANSVERSE | |
| | | | Colon | |
| 21 | 200602_48 | E200602018 | Paraffin | Brachyspira aalborgi |
| | | | embedded | DNA detected with |
| | | | tissue Biopsy | 16S rRNA gene |
| | | | CECUM | primer set. |
| 22 | 210903_083 | | Not found; | |
| | | | omitted | |
| 23 | 220103_049 | | Not found; | |
| | | | omitted | |
| 24 | 220513_070 | E220515043 | Bronchoalveolar | Haemophilus |
| | | | Lavage Lung, | influenzae |
| | | | Right Middle | Evaluating sample |
| | | | Lobe | for reflexive NGS |
| | | | | testing |
| 25 | 220506_076 | E220506125 | Bronchoalveolar | Multiple bacterial |
| | | | Lavage Lung, | DNA templates |
| | | | Right | detected |
| 26 | 220116_001 | E220116041 | Tissue | Trichomonas |
| | | | Bronchoalveolar | species (T. pallidum |
| | | | Lavage | Assay) |
| 27 | 210611_39 | | Not found; | |
| | | | omitted | |
| 28 | 220520_023 | E220520049 | Urine | Mycoplasma |
| | | | | genitalium Detected |
| 29 | 210830_057 | E210830060 | Tissue Paraffin | Penicillium species |
| | | | embedded | (Path report |
| | | | tissue Aorta | interest) |
| | | | Resection | |
| 30 | 200206_15 | E200206043 | Paraffin | Streptococcus |
| | | | embedded | dysgalactiae |
| | | | tissue Right | |
| | | | Nasal Mass | |
| 31 | 210623_21 | E210623020 | Tissue Penile | Candida albicans/ |
| | | | Ulcer | Staphylococcus |
| | | | | epidermidis; NGS: |
| | | | | [[Major |
| | | | | abundance]: |
| | | | | Staphylococcus |
| | | | | epidermidis |
| | | | | [Moderate |
| | | | | abundance]: |
| | | | | Enterococcus |
| | | | | faecalis, |
| | | | | Staphylococcus |
| | | | | haemolyticus, |
| | | | | Streptococcus |
| | | | | parasanguinis, |
| | | | | Streptococcus |
| | | | | salivarius group |
| 32 | 210901_102 | | Not found; | |
| | | | omitted | |
| 33 | 220313_006 | | Not found; | |
| | | | omitted | |
| 34 | 220316_050 | E220316057 | Intervertebral | Neisseria |
| | | | Disc | gonorrhoeae (T. |
| | | | | pallidum Assay) |
| 35 | 220317_008 | E220317019 | Right Finger | Neisseria |
| | | | | gonorrhoeae (T. |
| | | | | pallidum Assay) |

Clinical Specimens Known Positive for T. pallidum by BCTDNA

| | | | | |
|---|---|---|---|---|
| 36 | 210617_45 | E210617049 | Tissue, Paraffin | Treponema |
| | | | embedded | pallidum |
| | | | Penile Lesion | |
| 37 | 210629_42 | E210629047 | Tissue, Paraffin | Treponema |
| | | | embedded Left | pallidum |
| | | | Lower Lip | |
| 38 | 220308_068 | E220309155 | Paraffin | Treponema |
| | | | embedded | pallidum |
| | | | tissue Left | |
| | | | Inguinal Lymph | |
| | | | Node | |
| 39 | 211223_094 | E211226068 | Tissue, Paraffin | Treponema |
| | | | embedded | pallidum |
| | | | Umbilical Cord | |
| | | | and Membrane | |
| 40 | 211226_005 | E211226031 | Tissue FETAL | Treponema |
| | | | SPLEEN | pallidum |

Clinical Cases with History or Suspicion of Syphilis

| | | | | |
|---|---|---|---|---|
| 41 | 220124_019 | E220124050 | Tissue, Paraffin | Corynebacterium |
| | | | embedded Left | tuberculostearicum, |
| | | | Leg Skin | client wanted to |
| | | | | R/O T. pallidum |
| 42 | 220106_022 | E220106024 | Fluid Left Eye | Negative: client |
| | | | Vitreous Fluid | wanted to R/O T. |
| | | | | pallidum |
| 43 | 210829_049 | E210829051 | Tissue Paraffin | NEGATIVE bacterial, |
| | | | embedded | AFB, fungal PCRs; |
| | | | tissue Lymph | Path report |
| | | | Node Left Groin | indicated interest in |
| | | | | syphilis |
| 44 | 200707_85 | E200707088 | Biopsy Right | No bacterial DNA, |
| | | | Elbow | including |
| | | | RECEIVED | Treponema |
| | | | ROOM TEMP | pallidum, detected |
| | | | | with 16S rDNA |
| | | | | primer set |
| 45 | 200213_32 | | Not found; | |
| | | | omitted | |
| 46 | 200114_40 | E200114043 | Fluid Vitreous | Negative: client |
| | | | Fluid Eye, Right | wanted to R/O |
| | | | | Syphillus |
| 47 | 200108_72 | E200109041 | Tissue Liver | Negative: client |
| | | | | wanted to R/O T. |
| | | | | pallidum |

Test Cases with Known or Suspected T pallidum below Limit of Detection for BCTDNA

| | | | | |
|---|---|---|---|---|
| 48 | 220529_064 | E220613042 | Paraffin | Multiple bacterial |
| | | | embedded | DNA templates |
| | | | tissue Lymph | detected |
| | | | Node Cervical | |
| 49 | 220412_075 | E220413083 | PET Skin punch | Not applicable None |
| | | | Biopsy | detected |
| 50 | 200616_1 | E200616001 | Paraffin | MULTE-NGSLTQ: |
| | | | embedded | collected for |
| | | | tissue Arm, | pathology report of |
| | | | Right Skin Ulcer | secondary Syphilis |
| 51 | 211006_063 | | Not found; | |
| | | | omitted | |
| 52 | 220616_095 | E220621099/ | Paraffin | MULT-NGSLTQ |
| | | E220621100 | embedded | |
| | | | tissue Anal | |
| | | | Ulcer | |

Additional Cases

| | | | | |
|---|---|---|---|---|
| 53 | 220621_014 | E220621009 | Lung, lower | T. denticola, |
| | | | right lobe | Treponema sp |
| 54 | 220624_059 | E220626081 | Parrafin | IHC positive for T. |
| | | | embedded | pallidum (per path |
| | | | tissue, right | report); T. |
| | | | medial thigh | refringens by NGS |
| | | | mass | |
| 55 | 220625_021 | E220626035 | Pleural fluid | T. lecithinolyticum |
| 56 | 220625_049 | E220626033 | Right fibula | S. agalactiae |
| 57 | 220427_111 | E220428099 | Placenta tissue | Mycoplasma |
| | | | | hominis |
| 58 | 220128_012 | E220128023 | Urine | Ureaplasma parvum |
| | | | | or U. urealyticum |
| | | | | (MPLDNA) |
| 59 | 220113_057 | E220113062 | Vaginal swab | Ureaplasma parvum |
| | | | | or U. urealyticum |
| | | | | (MPLDNA) |
| 60 | 220216_020 | E220216030 | Vaginal swab | Ureaplasma parvum |
| | | | | or U. urealyticum |
| | | | | (MPLDNA) |
| 61 | 220207_012 | E220207014 | Paraffin | C. glabrata, R. |
| | | | embedded | oryzae complex, |
| | | | tissue, Right | Lactobacillus |
| | | | inferior septum | delbrueckii, L |
| | | | | rhamnosus |
| 62 | 220215_055 | E220215080 | BAL | C. glabrata & C |
| | | | | tropicalis; S aureus, |
| | | | | P aeruginosa |
| 63 | 220601_069 | E220608050 | Epidural tissue | Aspergillus |
| | | | | fumigatus |
| 64 | TP pertenue - | | extracted DNA | T. pallidum subsp |
| | Gauthier | | vs cell pellet | pertenue |
| | (neat) | | | |
| 65 | TP | | extracted DNA | T. pallidum subsp |
| | endemicum - | | vs cell pellet | endemicum |
| | IraqB (neat) | | | |
| 66 | 220110_047 | E220111037 | Paraffin | Tropheryma sp. |
| | | | embedded | related to T. |
| | | | tissue, | whipplei |
| | | | duodenum | |
| | | | biopsy | |
| 67 | TP pertenue - | | | |
| | Gauthier | | | |
| | (1:10 in | | | |
| | residual | | | |
| | patient | | | |
| | negative) | | | |
| 68 | TP | | | |
| | endemicum - | | | |
| | IraqB (1:10 in | | | |
| | residual | | | |
| | patient | | | |
| | negative) | | | |
| 69 | TP pertenue - | | | |
| | Gauthier | | | |
| | (1:100 in | | | |
| | residual | | | |
| | patient | | | |
| | negative) | | | |
| 70 | TP | | | |
| | endemicum - | | | |
| | IraqB (1:100 | | | |
| | in residual | | | |
| | patient | | | |
| | negative) | | | |

### B. Results

### B1. Discriminatory Power of Amplified Region (Analytical Specificity)

B1.1. Synthetic gBlock Controls

In addition to the cultured strains and clinical specimens shown in Table 3, a panel of thirty synthetic DNA controls (500 bp gBlocks, Integrated DNA Technologies) were also included in the analysis. These synthetic controls represented the 23S IRNA D1 domain from a variety of non-pallidum treponemal species, organisms found at oropharyngeal, genitourinary, and gastrointestinal mucosal sites (Table 4), and T. pallidum, which was used as the positive control. Non-pallidum Treponema spp. with ≥70% identity by BLAST to any assay primer or the target analyte were also included. The rabbit pathogen T. paraluiscuniculi has known 100% nucleotide identity to T. pallidum at rRNA and other loci and was excluded due to clinical irrelevance and predicted cross-reactivity. For the remaining organisms, organisms that were difficult to cultivate or could cause other sexually transmitted infections were prioritized. These synthetic controls were tested by the LAMP assay along with the positive control gBlock, extracted DNA from cultured isolates, and non-template controls (NTCs). Human gDNA (30-50 ng) was included in each reaction to simulate patient matrix.

TABLE 4
Organisms Represented in gBlock Specificity Controls

| Named | | Unnamed | | Non- | |
|---|---|---|---|---|---|
| Treponema | | Treponema | | Treponema | |
| spp. | Notes | spp. | Notes | spp. | Notes |
| T. phagedenis | Bovine / | Treponema sp. | Human | Chlamydia | CP042717 |
| | ovine | OMZ838 | oral | trachomatis | |
| | | | | KU2043 | |
| T. vincentii | Human oral | Treponema sp. | Human | C. pneumoniae | LN847058 |
| | | MarseilleQ4132 | oral | DC9 | |
| T. primitia | Termite gut | Treponema sp. | Human | Brachyspira | NR_076972 |
| | | isolate nC27 | gut | pilosicoli | animal & human |
| | | | | | pathogen |
| T. medium | Human oral | | | Brachyspira | SAYD01000018.1 |
| | | | | aalborgi | |
| T. pedis | Bovine | | | Neisseria | NR_103957 |
| | | | | gonorrheae | |
| T. putidum | Human oral | | | Neisseria | NR_103955 |
| | | | | meningitidis | |
| T. parvum | Human oral | | | Haemophilus | NR_076207 |
| | commensal | | | ducreyii | |
| T. denticola | Human oral | | | Mycoplasma | NR_077054 |
| | | | | genitalium | |
| T. peruense | Human GI | | | Ureaplasma | NR_103078 |
| | commensal | | | urealyticum | |
| T. socranskii | Human oral | | | Gardnerella | NR_076711 |
| | | | | vaginalis | |
| T. rectale | Bovine | | | | |
| | rectum | | | | |
| T. | Termite gut | | | | |
| azotonutricium | | | | | |
| T. ruminis | Bovine, | | | | |
| | from rumen | | | | |
| T. caldarium | Avirulent, | | | | |
| | thermophilic | | | | |
| T. | Animal & | | | | |
| succinifaciens | rare human | | | | |
| | commensal | | | | |
| T. | Animal | | | | |
| brennaborense | pathogen | | | | |

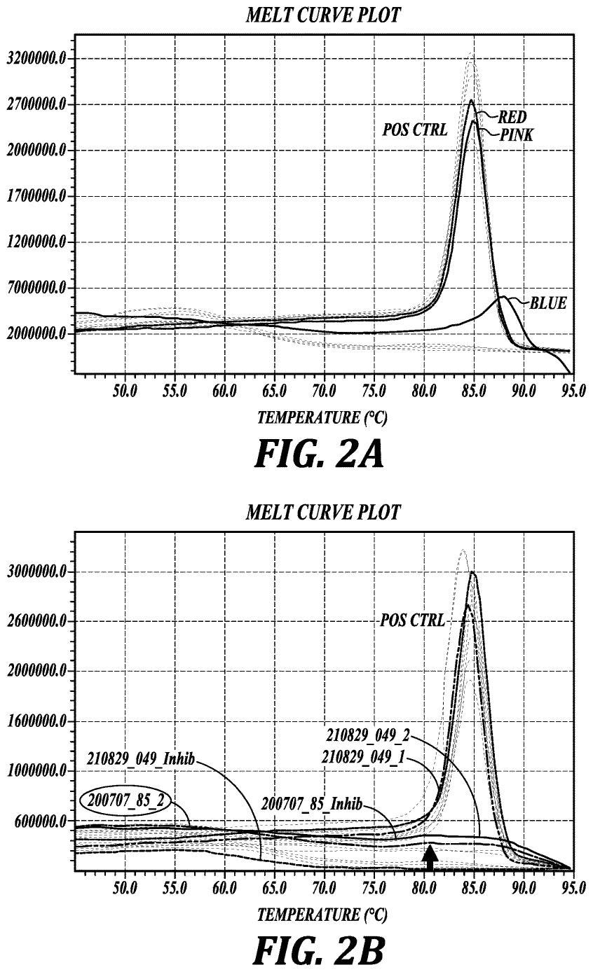

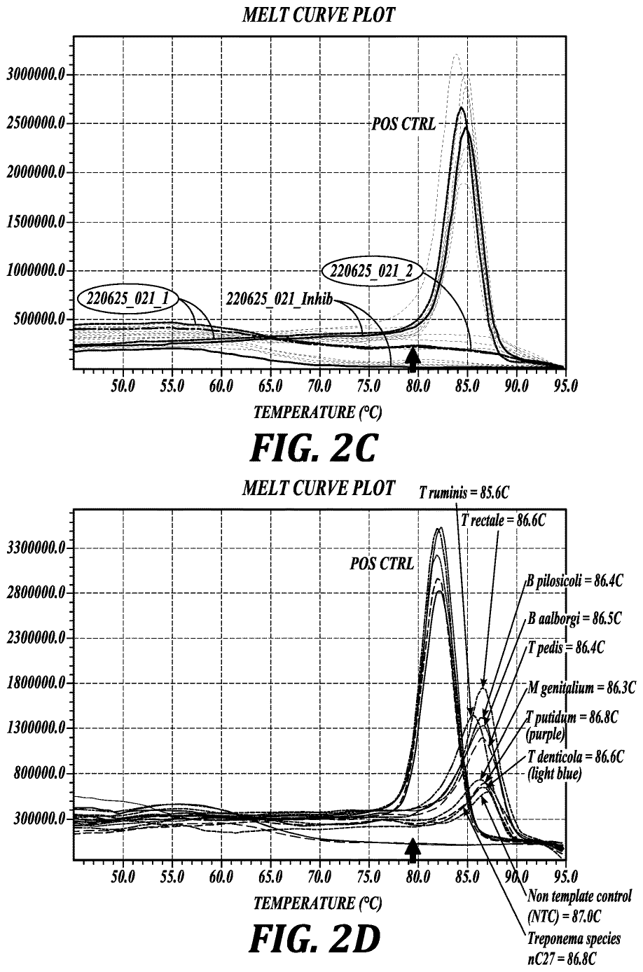

Results from three different experiments comprising five technical replicates, including control and off-target amplifications, are summarized in Table 5. Each experiment was performed on a different day by one of two operators. In melt curve analysis, the positive control melting temperature (Tₘ) was 82-84° C., while rare off-target amplification could be readily distinguished by a Tₘ>85-86° C. and by its different shape or intensity (see FIGS. 2A-2D which demonstrate specificity in clinical samples and evaluation for reaction inhibition). Full results are reported in Table 7. The first experiment was run for a duration of 60 min and accounted for eleven of thirteen off-target amplifications; six of those eleven had C_{T}>40. Based on the consistently fast amplification of target and late off-target amplification reaction, run time was shortened to 40 min. Because rare off-target amplification can be readily distinguished by melt curve analysis, it was concluded that the assay does not produce false-positives from tested synthetic 23S dsDNA.

TABLE 5
Summary of Specificity Control gBlock Amplifications

| | % Replicates | | |
|---|---|---|---|
| | Positive | Average | Average |
| Target | (N = 5) | C_{T} | Tₘ |
| T. pallidum | 100% | 15.0 | 84.2 |
| (positive control) | | | |
| T. pedis | 40% | 32.9 | 86.4 |
| T. putidum | 20% | 31.4 | 86.8 |
| T. denticola | 20% | 47.0 | 86.6 |
| T. rectale | 20% | 37.8 | 86.6 |
| T. ruminis | 20% | 43.6 | 85.6 |
| Treponema sp. | 20% | 33.5 | 87.3 |
| Marseille-Q4132¹ | | | |
| Treponema sp. nC27 | 20% | 30.4 | 86.8 |
| C. pneumoniae¹ | 20% | 31.4 | 86.7 |
| Brachyspira pilosicoli | 20% | 42.0 | 86.4 |
| B. aalborgi | 20% | 40.8 | 86.5 |
| M. genitalium | 20% | 48.4 | 86.3 |
| T. pallidum UW228B | 100% | 14.4 | 83.3 |
| (20-25 copies) | (N = 4) | | |
| T. pallidum UW228B | 100% | 14.6 | 83.7 |
| (10-12 copies) | (N = 6) | | |
| T. pallidum UW99B | 50% | 15.9 | 84.5 |
| (7.5-15 copies) | (N = 6) | | |
| T. pallidum Nichols | 100% | 15.3 | 84.3 |
| (5-10 copies) | (N = 6) | | |
| Non-template control | 17% | 41.4 | 87 |
| | (N = 6) | | |

¹The positive control Tₘ was 82.2 °C for the first run, which had a longer run time of 60 min and included one off-target amplification for all targets except Treponema sp. Marseille-Q4132 and C. pneumoniae, which were detected in the second run.

B1.2. Reactivity with Human DNA

Cross-reactivity with human DNA was evaluated by spiking in human gDNA into all reactions (N=151) in the gBlock specificity experiments, including NTCs. Cross-reactivity with human DNA was also interrogated by sequencing representative “positive” results and any off-target amplifications detected from residual clinical cases and reproducibility studies (see Table 8 which demonstrates precision and accuracy). One NTC replicate each from gBlock and residual clinical case experiments produced late amplifying off-target results (C_{T}>30) with a Tₘ~3° C. greater than the positive control (see FIG. 2A, Tables 9 and 10 which demonstrates specificity in clinical samples and evaluation for reaction inhibition). No off-target amplification of human DNA was observed in any replicate. Because rare off-target amplification can be readily distinguished by melt curve analysis, it was concluded that the assay does not produce false-positives from human gDNA.

### B1.3. Specificity in Clinical Samples and Evaluation for Reaction Inhibition

Forty-six residual clinical samples in which T. pallidum was not detected were interrogated by this assay. 5 mcL of each residual eluate was used as template in technical duplicates, and 2.5 mcL were mixed 1:1 with the positive gBlock control (625 copies per reaction) to assess inhibition due to matrix effects. Reactions were performed in 96-well plates; each plate included positive controls comprising gBlock at 1,250 copies per reaction and TP Nichols gDNA at 10 copies per reaction, as well as NTCs (N=6) as negative controls. The specimens included six cases in which the laboratory had been asked to “rule out” T. pallidum infection and in which this target organism was not detected by 16S bacterial PCR.

All forty-six residual clinical specimens tested as specificity controls were tested negative by the LAMP assay. None of the forty-six specimens produced any amplification in any replicate. Although the QuantStudio software called two specimens as positive with melt curve Tₘ's significantly different from the positive controls (eluate #44 has two replicates with Tₘ=80.5° C.; eluate #55 has two replicates with Tₘ=79.9° C.), visual inspection of the melt curves demonstrated no visible peak. The results were thus determined as negative. One NTC produced signal during melt curve analysis but with a Tₘ=87° C. (FIG. 2A). All Tₘ's were readily distinguishable from the positive controls by both automated instrument analysis and manual review. Sequencing of the off-target amplified products demonstrated low-quality, off-target sequences (see Section B5: Evaluation of Sequencing).

No inhibition was observed in any inhibition controls. Table 6 summarizes all organisms and sequence variants represented in the clinical specimens. Full results are shown in Table 8 and a complete list of organisms represented is shown in Table 9.

TABLE 6
Summary of Organisms Represented
in All Residual Clinical Samples

| Organism/Category | Count |
|---|---|
| Total bacterial species | 117 |
| Total bacterial SV's | 237 |
| Total fungal species | 9 |
| Total fungal SV's | 9 |
| Total Trichomonas spp.¹ | 3 |
| T. vaginalis | 1 |
| Total Treponema spp. | 9 |
| Total Treponema spp. SV's | 29 |
| Total Treponemataceae | 4 |
| Total Spirochaetales | 1 |
| Total T. pallidum subsp. | 6 |
| pallidum | |
| Total T. pallidum other | 2² |
| subsp. | |

¹Two organisms reported as Trichomonas sp. had ~96% nucleotide identity to published (M19224) and unpublished (MN178558) records for T. vaginalis in NCBI nr/nt; the next nearest matching record by blast was Pentatrichomonas hominis at ~83% nucleotide identity.
²T. pallidum subsp. endemicum (Iraq B) and T. pallidum subsp. pertenue (Gauthier) were tested at three different dilations, each in technical duplicate and with an inhibition control.

nearest matching record by blast was Pentatrichomonas hominis at ~83% nucleotide identity.
2 T. pallidum subsp. endemicum (Iraq B) and T. pallidum subsp. pertenue (Gauthier) were tested at three different dilutions, each in technical duplicate and with an inhibition control.

TABLE 7
Results of gBlock Control Experiments
Run 1

| Sample | Ct1 | Tm1 | Date | Operator |
|---|---|---|---|---|
| T. pallidum | 17.6* | 82.2* | Jun. 2, 2022 | @joshuaal |
| T. phagedenis | 38 | 55.7 | Jun. 2, 2022 | @joshuaal |
| T. vincentii | 42.4 | 55.1 | Jun. 2, 2022 | @joshuaal |
| T. primitia | 36.3 | 55.5 | Jun. 2, 2022 | @joshuaal |
| T. medium | 35 | 55.3 | Jun. 2, 2022 | @joshuaal |
| T. pedis | 37.8** | 86.7** | Jun. 2, 2022 | @joshuaal |
| T. putidum | 31.4** | 86.8** | Jun. 2, 2022 | @joshuaal |
| T. parvum | 29.3 | 55.9 | Jun. 2, 2022 | @joshuaal |
| T. denticola | 47** | 86.6** | Jun. 2, 2022 | @joshuaal |
| T. peruense | 52 | 56.1 | Jun. 2, 2022 | @joshuaal |
| T. socranskii | Undetermined | 55.3 | Jun. 2, 2022 | @joshuaal |
| T. rectale | 37.8** | 86.6** | Jun. 2, 2022 | @joshuaal |
| T. azonutricium | 43.1 | 55.5 | Jun. 2, 2022 | @joshuaal |
| T. ruminis | 43.6** | 85.6** | Jun. 2, 2022 | @joshuaal |
| T. caldarium | 29.4 | 55.7 | Jun. 2, 2022 | @joshuaal |
| T. succinifaciens | 57.7 | 55.1 | Jun. 2, 2022 | @joshuaal |
| T. brennaborense | 38.8 | 55.5 | Jun. 2, 2022 | @joshuaal |
| Treponema sp. OMZ838 | 50.4 | 55.9 | Jun. 2, 2022 | @joshuaal |
| Treponema sp. Marseille-Q4132 | 47.4 | 54.6 | Jun. 2, 2022 | @joshuaal |
| Treponema sp. nC27 | 30.4** | 86.8** | Jun. 2, 2022 | @joshuaal |
| Chlamydia trachomatis | 40 | 55.5 | Jun. 2, 2022 | @joshuaal |
| C. pneumoniae | 46.4 | 55.9 | Jun. 2, 2022 | @joshuaal |
| Brachyspira pilosicoli | 42** | 86.4** | Jun. 2, 2022 | @joshuaal |
| Neisseria gonorrheae | 41.6 | 55.7 | Jun. 2, 2022 | @joshuaal |
| N. meningitidis | 44.9 | 55.3 | Jun. 2, 2022 | @joshuaal |
| Haemophilus ducreyii | 27.7 | 55.7 | Jun. 2, 2022 | @joshuaal |
| Mycoplasma genitalium | 48.4** | 86.3** | Jun. 2, 2022 | @joshuaal |
| Ureaplasma urealyticum | 27.4 | 54.2 | Jun. 2, 2022 | @joshuaal |
| Gardnerella vaginalis | 32.6 | 55.7 | Jun. 2, 2022 | @joshuaal |
| Brachyspira aalborgi | 40.8** | 86.5** | Jun. 2, 2022 | @joshuaal |
| UW228B (25 copies/rxn) | 14.2* | 82.1* | Jun. 2, 2022 | @joshuaal |
| UW228B (25 copies/rxn) | 17.5* | 82.1* | Jun. 2, 2022 | @joshuaal |
| UW228B (12 copies/rxn) | 18.6* | 81.9* | Jun. 2, 2022 | @joshuaal |
| UW228B (12 copies/rxn) | 13.7* | 81.9* | Jun. 2, 2022 | @joshuaal |
| UW228B (20 copies/rxn) | | | | |
| UW228B (20 copies/rxn) | | | | |
| UW228B (10 copies/rxn) | | | | |
| UW228B (10 copies/rxn) | | | Jun. 2, 2022 | @joshuaal |
| UW99B (E02) (15 copies/rxn) | | | Jun. 2, 2022 | @joshuaal |
| UW99B (E02) (7.5 copies/rxn) | | | Jun. 2, 2022 | @joshuaal |
| Nichols (10 copies/rxn) | | | Jun. 2, 2022 | @joshuaal |
| Nichols (5 copies/rxn) | | | Jun. 2, 2022 | @joshuaal |
| NTC | Undetermined | 55.7 | Jun. 2, 2022 | @joshuaal |
| NTC | 41.4** | 87** | Jun. 2, 2022 | @joshuaal |
| TPAL gblock (add'l repeat) | | | | |

| | | | | | | |
|---|---|---|---|---|---|---|
| Sample | Ct1 | Tm1 | Ct2 | Tm2 | Date | Operator |

Run 2

| | | | | | | |
|---|---|---|---|---|---|---|
| T. pallidum | 20* | 84.7* | 10.9* | 84.4* | Jun. 9, 2022 | @rkwon2 |
| T. phagedenis | Undetermined | 55.1 | Undetermined | 54.9 | Jun. 9, 2022 | @rkwon2 |
| T. vincentii | Undetermined | 55.1 | Undetermined | 55.1 | Jun. 9, 2022 | @rkwon2 |
| T. primitia | Undetermined | 55.3 | Undetermined | 55.1 | Jun. 9, 2022 | @rkwon2 |
| T. medium | 35 | 52.8 | Undetermined | 54.9 | Jun. 9, 2022 | @rkwon2 |
| T. pedis | 27.9** | 86.1** | Undetermined | 55.1 | Jun. 9, 2022 | @rkwon2 |
| T. putidum | Undetermined | 54.9 | Undetermined | 55.3 | Jun. 9, 2022 | @rkwon2 |
| T. parvum | Undetermined | 55.5 | Undetermined | 55.3 | Jun. 9, 2022 | @rkwon2 |
| T. denticola | 19.2 | 55.7 | Undetermined | 54.9 | Jun. 9, 2022 | @rkwon2 |
| T. peruense | 10 | 55.5 | Undetermined | 55.1 | Jun. 9, 2022 | @rkwon2 |
| T. socranskii | Undetermined | 54.2 | Undetermined | 55.3 | Jun. 9, 2022 | @rkwon2 |
| T. rectale | Undetermined | 54.2 | Undetermined | 55.1 | Jun. 9, 2022 | @rkwon2 |
| T. azonutricium | Undetermined | 55.1 | Undetermined | 55.1 | Jun. 9, 2022 | @rkwon2 |
| T. ruminis | Undetermined | 55.5 | Undetermined | 55.5 | Jun. 9, 2022 | @rkwon2 |
| T. caldarium | Undetermined | 55.1 | Undetermined | 55.7 | Jun. 9, 2022 | @rkwon2 |
| T. succinifaciens | Undetermined | 55.3 | Undetermined | 55.1 | Jun. 9, 2022 | @rkwon2 |
| T. brennaborense | Undetermined | 55.1 | 7.8 | 55.1 | Jun. 9, 2022 | @rkwon2 |
| Treponema sp. OMZ838 | Undetermined | 55.5 | Undetermined | 55.3 | Jun. 9, 2022 | @rkwon2 |
| Treponema sp. Marseille-Q4132 | 33.5** | 87.3** | Undetermined | 55.3 | Jun. 9, 2022 | @rkwon2 |
| Treponema sp. nC27 | 25.2 | 55.1 | Undetermined | 55.1 | Jun. 9, 2022 | @rkwon2 |
| Chlamydia trachomatis | 11.8 | 55.3 | Undetermined | 55.1 | Jun. 9, 2022 | @rkwon2 |
| C. pneumoniae | Undetermined | 55.3 | 31.4** | 86.7** | Jun. 9, 2022 | @rkwon2 |
| Brachyspira pilosicoli | Undetermined | 54.9 | Undetermined | 55.3 | Jun. 9, 2022 | @rkwon2 |
| Neisseria gonorrheae | Undetermined | 55.3 | Undetermined | 55.1 | Jun. 9, 2022 | @rkwon2 |
| N. meningitidis | Undetermined | 55.5 | Undetermined | 54.9 | Jun. 9, 2022 | @rkwon2 |
| Haemophilus ducreyii | Undetermined | 54.9 | Undetermined | 55.3 | Jun. 9, 2022 | @rkwon2 |
| Mycoplasma genitalium | Undetermined | 55.1 | Undetermined | 55.3 | Jun. 9, 2022 | @rkwon2 |
| Ureaplasma urealyticum | Undetermined | 55.5 | Undetermined | 55.5 | Jun. 9, 2022 | @rkwon2 |
| Gardnerella vaginalis | 33.2 | 55.3 | Undetermined | 55.3 | Jun. 9, 2022 | @rkwon2 |
| Brachyspira aalborgi | Undetermined | 55.1 | Undetermined | 55.3 | Jun. 9, 2022 | @rkwon2 |
| UW228B (25 copies/rxn) | | | | | | |
| UW228B (25 copies/rxn) | | | | | | |
| UW228B (12 copies/rxn) | | | | | | |
| UW228B (12 copies/rxn) | | | | | | |
| UW228B (20 copies/rxn) | 12.9* | 84.4* | 12.9* | 84.4* | Jun. 9, 2022 | @rkwon2 |
| UW228B (20 copies/rxn) | | | | | | |
| UW228B (10 copies/rxn) | | | | | | |
| UW228B (10 copies/rxn) | 11.8* | 84.2* | 13.8* | 84.6* | Jun. 9, 2022 | @rkwon2 |
| UW99B (E02) (15 copies/rxn) | Undetermined*** | 54.9*** | 15* | 84* | Jun. 9, 2022 | @rkwon2 |
| UW99B (E02) (7.5 copies/rxn) | Undetermined*** | 54.9*** | 13.2* | 84.8* | Jun. 9, 2022 | @rkwon2 |
| Nichols (10 copies/rxn) | 11.6* | 84.2* | 14.1* | 84.2* | Jun. 9, 2022 | @rkwon2 |
| Nichols (5 copies/rxn) | 38.7* | 84.2* | 12.9* | 84* | Jun. 9, 2022 | @rkwon2 |
| NTC | Undetermined | 55.3 | Undetermined | 54.9 | Jun. 9, 2022 | @rkwon2 |
| NTC | | | | | | |
| TPAL gblock (add'l repeat) | 13.4* | 84.5* | 12.8* | 84.9* | Jun. 9, 2022 | @rkwon2 |

Run 3

| | | | | | | |
|---|---|---|---|---|---|---|
| T. pallidum | 13.166534* | 84.86041* | 13.556964* | 84.86041* | Jun. 24, 2022 | @rkwon2 |
| T. phagedenis | 29.465935 | 54.24643 | 20.355995 | 54.86282 | Jun. 24, 2022 | @rkwon2 |
| T. vincentii | 22.018667 | 54.4569 | 29.714945 | 54.4569 | Jun. 24, 2022 | @rkwon2 |
| T. primitia | Undetermined | 55.07358 | 20.901041 | 54.86802 | Jun. 24, 2022 | @rkwon2 |
| T. medium | 22.294157 | 55.070312 | 21.114727 | 54.24836 | Jun. 24, 2022 | @rkwon2 |
| T. pedis | 24.495785 | 55.68678 | 18.515314 | 54.042873 | Jun. 24, 2022 | @rkwon2 |
| T. putidum | 19.067091 | 55.26554 | Undetermined | 54.444386 | Jun. 24, 2022 | @rkwon2 |
| T. parvum | 20.800276 | 55.060253 | 21.1543 | 55.060253 | Jun. 24, 2022 | @rkwon2 |
| T. denticola | 24.823921 | 54.652985 | 20.088753 | 54.85834 | Jun. 24, 2022 | @rkwon2 |
| T. peruense | 36.807396 | 54.242264 | 26.573126 | 55.47442 | Jun. 24, 2022 | @rkwon2 |
| T. socranskii | 32.58513 | 54.651474 | 33.239616 | 54.85681 | Jun. 24, 2022 | @rkwon2 |
| T. rectale | 25.252148 | 55.062153 | 35.52915 | 54.85681 | Jun. 24, 2022 | @rkwon2 |
| T. azonutricium | Undetermined | 54.04097 | 22.826328 | 54.24643 | Jun. 24, 2022 | @rkwon2 |
| T. ruminis | 20.903599 | 53.630043 | Undetermined | 54.24643 | Jun. 24, 2022 | @rkwon2 |
| T. caldarium | 26.733482 | 54.66246 | 35.173817 | 54.045776 | Jun. 24, 2022 | @rkwon2 |
| T. succinifaciens | 26.74697 | 54.045776 | 35.517094 | 52.812412 | Jun. 24, 2022 | @rkwon2 |
| T. brennaborense | 24.80702 | 55.070312 | Undetermined | 54.24836 | Jun. 24, 2022 | @rkwon2 |
| Treponema sp. OMZ838 | 23.450027 | 54.24836 | 28.520811 | 54.24836 | Jun. 24, 2022 | @rkwon2 |
| Treponema sp. Marseille-Q4132 | 19.452154 | 54.033806 | Undetermined | 53.828518 | Jun. 24, 2022 | @rkwon2 |
| Treponema sp. nC27 | 18.440182 | 54.033806 | Undetermined | 53.828518 | Jun. 24, 2022 | @rkwon2 |
| Chlamydia trachomatis | Undetermined | 54.85834 | Undetermined | 54.85834 | Jun. 24, 2022 | @rkwon2 |
| C. pneumoniae | Undetermined | 54.036903 | 22.739199 | 54.85834 | Jun. 24, 2022 | @rkwon2 |
| Brachyspira pilosicoli | Undetermined | 54.03546 | 36.22232 | 54.651474 | Jun. 24, 2022 | @rkwon2 |
| Neisseria gonorrheae | Undetermined | 53.830124 | 27.71835 | 54.03546 | Jun. 24, 2022 | @rkwon2 |
| N. meningitidis | 35.017765 | 55.068283 | Undetermined | 54.24643 | Jun. 24, 2022 | @rkwon2 |
| Haemophilus ducreyii | 21.047583 | 55.47921 | Undetermined | 54.24643 | Jun. 24, 2022 | @rkwon2 |
| Mycoplasma genitalium | Undetermined | 55.279144 | 25.468111 | 54.4569 | Jun. 24, 2022 | @rkwon2 |
| Ureaplasma urealyticum | 26.229784 | 55.89583 | 24.71506 | 54.045776 | Jun. 24, 2022 | @rkwon2 |
| Gardnerella vaginalis | 20.077238 | 54.65934 | 18.490255 | 53.837387 | Jun. 24, 2022 | @rkwon2 |
| Brachyspira aalborgi | 24.529245 | 54.864826 | 24.089191 | 54.45385 | Jun. 24, 2022 | @rkwon2 |
| UW228B (25 copies/rxn) | | | | | | |
| UW228B (25 copies/rxn) | | | | | | |
| UW228B (12 copies/rxn) | | | | | | |
| UW228B (12 copies/rxn) | | | | | | |
| UW228B (20 copies/rxn) | | | | | | |
| UW228B (20 copies/rxn) | | | | | | |
| UW228B (10 copies/rxn) | | | | | | |
| UW228B (10 copies/rxn) | 18.014996* | 85.032425* | 11.900635* | 84.82714* | Jun. 24, 2022 | @rkwon2 |
| UW99B (E02) (15 copies/rxn) | | | | | | |
| UW99B (E02) (7.5 copies/rxn) | 17.596682* | 84.84079* | 17.840641*** | 55.88514*** | Jun. 24, 2022 | @rkwon2 |
| Nichols (10 copies/rxn) | | | | | | |
| Nichols (5 copies/rxn) | 7.1358943* | 84.6308* | 7.1121* | 84.6308* | Jun. 24, 2022 | @rkwon2 |
| NTC | 27.736013 | 55.060253 | 23.853403 | 55.060253 | Jun. 24, 2022 | @rkwon2 |
| NTC | | | | | | |
| TPAL gblock (add'l repeat) | | | | | | |

*indicates successful amplification of positive controls with characteristic melt curves.
**indicate unexpected amplification with a melt curve readily distinguishable from both T. pallidum gDNA and gBlock positive controls.
***indicate failure of a positive control (UW99B, lysate E02). Copies per reaction: T. pallidum gBlock control at 1,000-1,250; UW99B, UW228B, and TP Nichols as indicated.

TABLE 8
Full Results of Residual Clinical Sample Amplifications

| | | | | | | | | gBlock | | Nichols | | Neg | |
|---|---|---|---|---|---|---|---|---|---|---|---|---|---|
| Study | | Duplicate 1 | | Duplicate 2 | | Inhib | | 2.5E2 | | 1:200K | | control | |
| ID | MDXID | Ct | Tm | Ct | Tm | Ct | Tm | Ct | Tm | Ct | Tm | Ct | Tm |
| TEST THESE FIRST: Non-pallidum Treponema specificity controls | | | | | | | | | | | | | |
| 1 | 220404_060 | — | 55.471 | — | 55.471 | 17.481 | 84.84 | 14.945 | 84.882 | 9.575 | 84.661 | — | 55.481 |
| 2 | 220428_040 | — | 55.684 | — | 55.479 | 16.175 | 84.882 | | | | | | |
| 3 | 220307_019 | — | 55.074 | — | 55.276 | 15.789 | 84.87 | | | | | | |
| 4 | 220224_102 | — | 55.068 | — | 55.479 | 14.98 | 84.88 | | | | | | |
| 5 | 220416_021 | — | 55.064 | — | 55.061 | 18.07 | 84.83 | | | | | | |
| 6 | 220420_068 | — | 54.657 | — | 54.247 | 15.105 | 84.879 | 18.853 | 84.858 | 12.377 | 84.858 | — | 54.663 |
| 7 | 220326_029 | — | 55.279 | — | 55.07 | 19.822 | 84.866 | | | | | | |
| 8 | 210823_13 | — | 54.855 | — | 55.266 | 17.298 | 84.841 | | | | | | |
| 9 | 201119_87 | — | 55.064 | — | 55.06 | 16.361 | 84.826 | | | | | | |
| 10 | 210922_068 | sample missing | | | | | | | | | | | |
| 11 | 220529_041 | — | 54.863 | — | 54.657 | 18.207 | 85.084 | | | | | | |
| 12 | 210821_68 | — | 54.868 | — | 54.865 | 17.725 | 84.866 | | | | | | |
| 13 | 211005_021 | sample missing | | | | | | | | | | | |
| 14 | 210609_81 | — | 54.65 | — | 54.855 | 19.505 | 84.841 | | | | | | |
| 15 | 211201_084 | — | 55.064 | — | 54.855 | 17.756 | 84.826 | | | | | | |
| 16 | 210818_49 | — | 54.452 | — | 54.247 | 20.091 | 84.879 | | | | | | |
| 17 | 220418_030 | — | 55.074 | — | 54.248 | 15.298 | 84.661 | | | | | | |
| 18 | 201107_31 | 36.603 | 54.855 | — | 54.855 | 19.885 | 84.636 | | | | | | |
| 19 | 200320_6 | — | 55.27 | — | 55.06 | 16.411 | 84.621 | | | | | | |
| TEST THESE SECOND: Non-Treponema specificity controls | | | | | | | | | | | | | |
| 20 | 210224_72 | — | 54.861 | — | 55.067 | 15.077 | 84.878 | 16.872 | 85.038 | 10.492 | 84.8 | 31.424 | 87.878 |
| 21 | 200602_48 | — | 55.074 | — | 55.067 | 13.543 | 84.853 | | | | | | |
| 22 | 210903_083 | sample missing | | | | | | | | | | | |
| 23 | 220103_049 | sample missing | | | | | | | | | | | |
| 24 | 220513_070 | 30.95 | 53.625 | 13.559 | 54.651 | 13.711 | 84.833 | | | | | | |
| 25 | 220506_076 | — | 55.062 | — | 55.054 | 13.483 | 84.595 | | | | | | |
| 26 | 220116_001 | — | 54.245 | — | 54.861 | 14.504 | 84.672 | | | | | | |
| 27 | 210611_39 | sample missing | | | | | | | | | | | |
| 28 | 220520_023 | — | 55.074 | — | 54.657 | 15.004 | 84.853 | | | | | | |
| 29 | 210830_057 | — | 53.83 | — | 54.446 | 14.968 | 84.833 | | | | | | |
| 30 | 200206_15 | — | 54.857 | — | 55.054 | 14.956 | 84.595 | | | | | | |
| 31 | 210623_21 | — | 54.656 | 13.514 | 55.272 | 14.48 | 84.672 | | | | | | |
| 32 | 210901_102 | sample missing | | | | | | | | | | | |
| 33 | 220313_006 | sample missing | | | | | | | | | | | |
| 34 | 220316_050 | — | 55.074 | — | 55.067 | 14.753 | 84.648 | | | | | | |
| 35 | 220317_008 | 32.446 | 55.062 | — | 55.062 | 16.829 | 84.628 | | | | | | |
| THIRD PRIORITY: Known positive cases detected by BCTDNA | | | | | | | | | | | | | |
| 36 | 210617_45 | 19.051 | 84.453 | 15.999 | 84.453 | 16.191 | 84.679 | 14.696 | 84.679 | 9.932 | 84.454 | — | 54.659 |
| 37 | 210629_42 | 10.245 | 84.473 | 10.342 | 84.454 | 10.788 | 84.454 | | | | | | |
| 38 | 220308_068 | 16.805 | 84.629 | 17.435 | 84.629 | 17.205 | 84.632 | | | | | | |
| 39 | 211223_094 | 13.258 | 84.837 | 12.914 | 84.823 | 13.23 | 84.618 | | | | | | |
| 40 | 211226_005 | 10.937 | 84.453 | 10.986 | 84.453 | 11.929 | 84.679 | | | | | | |
| FOURTH PRIORITY: Rule out cases (confirm rule | | | | | | | | | | | | | |
| out); history or suspicion of syphilis | | | | | | | | | | | | | |
| 41 | 220124_019 | — | 54.041 | — | 54.452 | 13.947 | 85.087 | 17.022 | 84.42 | 10.679 | 83.804 | — | 54.652 |
| 42 | 220106_022 | — | 54.252 | — | 54.248 | 16.151 | 85.071 | | | | | | |
| 43 | 210829_049 | — | 54.446 | — | 53.83 | 15.669 | 85.041 | | | | | | |
| 44 | 200707_85 | — | 53.214 | — | 80.511 | 18.4 | 85.027 | | | | | | |
| 45 | 200213_32 | sample missing | | | | | | | | | | | |
| 46 | 200114_40 | — | 54.452 | — | 54.863 | 15.699 | 84.881 | | | | | | |
| 47 | 200108_72 | — | 54.457 | — | 54.865 | 14.064 | 84.865 | | | | | | |
| FIFTH PRIORITY: Test Cases - possible or known | | | | | | | | | | | | | |
| TPAL below BCTDNA LOD | | | | | | | | | | | | | |
| 48 | 220529_064 | 15.219 | 84.625 | 15.426 | 84.83 | 14.523 | 84.835 | | | | | | |
| 40 | 220412_075 | — | 54.241 | — | 54.443 | 14.227 | 84.822 | | | | | | |
| 50 | 200616_1 | 16.854 | 84.861 | 19.091 | 84.861 | 14.634 | 84.881 | | | | | | |
| 51 | 211006_063 | sample missing | | | | | | | | | | | |
| 52 | 220616_095 | 34.532 | 54.252 | 38.813 | 54.659 | 17.484 | 84.865 | | | | | | |
| Additional Cases | | | | | | | | | | | | | |
| 53 | 220621_014 | — | 54.856 | — | 54.856 | 16.245 | 84.835 | | | | | | |
| 54 | 220624_059 | — | 53.42 | — | 54.649 | 18.26 | 84.822 | | | | | | |
| 55 | 220625_021 | 13.215 | 79.929 | — | 79.929 | 20.614 | 84.881 | | | | | | |
| 56 | 220625_049 | — | 54.663 | — | 54.865 | 15.69 | 84.66 | | | | | | |
| 57 | 220427_111 | 19.603 | 54.24 | — | 54.035 | 15.021 | 84.835 | | | | | | |
| 58 | 220128_012 | — | 54.652 | — | 54.854 | 16.775 | 84.822 | | | | | | |
| 59 | 220113_057 | — | 54.041 | — | 54.658 | 15.419 | 84.881 | | | | | | |
| 60 | 220216_020 | — | 54.869 | — | 54.454 | 18.787 | 84.66 | | | | | | |
| 61 | 220207_012 | — | 54.24 | — | 55.062 | 14.371 | 84.63 | | | | | | |
| 62 | 220215_055 | — | 54.652 | — | 54.443 | 15.101 | 84.822 | | | | | | |
| 63 | 220601_069 | — | 54.247 | — | 54.863 | 15.659 | 84.676 | | | | | | |
| 66 | 220110_047 | — | 54.869 | — | 54.248 | 15.237 | 84.66 | | | | | | |
| Additonal Subspecies | | | | | | | | | | | | | |
| 64 | TP | 7.515 | 84.88 | 7.561 | 84.662 | 7.776 | 84.868 | 17.901 | 85.052 | Not | | — | 46.643 |
| | pertenue | | | | | | | | | Done | | | |
| | (neat) | | | | | | | | | | | | |
| 65 | TP | 7.955 | 84.449 | 8.012 | 84.449 | 8.577 | 84.674 | | | | | | |
| | endemicum | | | | | | | | | | | | |
| | (neat) | | | | | | | | | | | | |
| 67 | TP | 9.013 | 84.835 | 9.053 | 84.835 | 9.236 | 84.847 | | | | | | |
| | pertenue - | | | | | | | | | | | | |
| | (1:10) | | | | | | | | | | | | |
| 68 | TP | 9.892 | 84.674 | 9.704 | 84.662 | 10.05 | 84.662 | | | | | | |
| | endemicum | | | | | | | | | | | | |
| | (1:10) | | | | | | | | | | | | |
| 69 | TP | 10.348 | 84.847 | 10.198 | 84.62 | 10.581 | 84.825 | | | | | | |
| | pertenue | | | | | | | | | | | | |
| | (1:100) | | | | | | | | | | | | |
| 70 | TP | 11.363 | 84.63 | 11.288 | 84.835 | 11.606 | 84.642 | | | | | | |
| | endemicum | | | | | | | | | | | | |
| | (1:100) | | | | | | | | | | | | |

TABLE 9
Complete List and Count of Organisms Represented

| | Total Count |
|---|---|
| Bacteria | |
| Actinobacteria, Class | 1 |
| Actinomyces israelii | 2 |
| Aggregatibacter actinomycetemcomitans | 1 |
| Aggregatibacter segnis | 1 |
| Alloprevotella spp | 1 |
| Alloprevotella tannerae | 6 |
| Annaerococcus murdochii | 1 |
| Bacteroidales, Order | 1 |
| Bacteroidetes, Phylum | 7 |
| Bacteroidia, Class | 1 |
| Brachyspira aalborgi | 2 |
| Campylobacter rectus | 4 |
| Campylobacter showae | 2 |
| Campylobacter spp | 1 |
| Campylobacter ureolyticus | 1 |
| Capnocytophaga granulosa | 1 |
| Capnocytophaga ledbetteri (RELT) | 1 |
| Clostridia, Class | 1 |
| Corynebacterium matruchotii | 1 |
| Corynebacterium tuberculostearicum | 2 |
| Dialister invisus | 1 |
| Dialister microaerophilus | 1 |
| Dialister pneumosintes | 3 |
| Enterococcus faecalis | 1 |
| Enterococcus faecium | 1 |
| Eubacteriales, Class | 1 |
| Eubacterium brachy | 3 |
| Eubacterium infirmum | 1 |
| Eubacterium nodatum | 1 |
| Filifactor alocis | 4 |
| Flavobacteriaceae, Family | 1 |
| Fretibacterium fastidiosum (RELT) | 1 |
| Fusobacteriaceae, Family | 1 |
| Fusobacterium gonadiaformans | 1 |
| Fusobacterium nucleatum | 15 |
| Fusobacterium sp | 4 |
| Gemella haemolysans or G. taiwanensis | 1 |
| Gemella morbillorum | 1 |
| Haemophilus influenzae | 4 |
| Haemophilus parainfluenzae | 1 |
| Kingella denitrificans | 1 |
| Lacticaseibacillus rhamnosus | 1 |
| Lactobacillus delbrueckii | 1 |
| Leptotrichiaceae, Family | 1 |
| Megasphaera massiliensis | 1 |
| MULTEM, no NGS | 4 |
| Mycoplasma genitalium | 1 |
| Mycoplasma hominis | 1 |
| Myocplasma faucium | 2 |
| Negative case | 6 |
| Negativicutes, Class | 1 |
| Neisseria bacilliformis | 1 |
| Neisseria gonorrhoeae | 2 |
| Neisseria mucosa | 2 |
| Odoribacter spp | 1 |
| Olegusella massiliensis | 1 |
| Parvimonas micra | 6 |
| Peptidiphaga spp | 1 |
| Peptoniphilus lacrimalis | 1 |
| Phocaeciola abscessus | 1 |
| Phocaeciola vulgatus | 1 |
| Porphorymonas bennonis | 2 |
| Porphorymonas endodontalis | 5 |
| Porphorymonas gingivalis | 8 |
| Porphorymonas somerae | 1 |
| Porphorymonas spp | 1 |
| Preovtella baroniae | 3 |
| Prevotella denticola | 1 |
| Prevotella histicola | 1 |
| Prevotella intermedia | 1 |
| Prevotella loescheii (RELT) | 3 |
| Prevotella melaninogenica | 3 |
| Prevotella multiformis | 1 |
| Prevotella oris | 2 |
| Prevotella pleuritidis | 2 |
| Proteobacteria, Phylum | 1 |
| Pseudomonas aeruginosa | 1 |
| Pseudomonas putida group | 1 |
| Pyramidobacter piscolens | 1 |
| Selenomonas sputigena | 1 |
| Snaethia turicensis | 1 |
| Snaethia vaginalis | 2 |
| Solobacterium moorei | 2 |
| Spirochaetales, Order | 1 |
| Staphylococcus aureus | 1 |
| Staphylococcus capitis or S. caprae or S. saccharolyticus | 1 |
| Staphylococcus epidermidis | 2 |
| Staphylococcus haemolyticus | 1 |
| Streptococcus agalactiae | 1 |
| Streptococcus anginosus | 1 |
| Streptococcus constellatus | 3 |
| Streptococcus dysgalactiae | 1 |
| Streptococcus intermedius | 2 |
| Streptococcus mitis | 1 |
| Streptococcus mitis group (not S. pneumoniae) | 2 |
| Streptococcus oralis | 1 |
| Streptococcus parasanguinis | 1 |
| Streptococcus pneumoniae or S pseudopneumoniae | 1 |
| Streptococcus salivarius group | 1 |
| Streptococcus sanguinis | 1 |
| Synergistetes, Phylum | 4 |
| Tannerella forsythia | 6 |
| Treponema denticola | 6 |
| Treponema lecithinolyticum | 2 |
| Treponema medium | 3 |
| Treponema pallidum | 6 |
| Treponema pallidum ENDEMICUM | 1 |
| Treponema pallidum PERTENUE | 1 |
| Treponema refringens | 1 |
| Treponema socranskii | 2 |
| Treponema spp | 7 |
| Treponemataceae, Family | 4 |
| Tropheryma whipplei (RELT) | 1 |
| Ureaplasma parvum | 1 |
| Ureaplasma parvum or U. urealyticum | 2 |
| Veillonella atypica | 1 |
| Veillonellaceae, Family | 1 |
| Fungi | |
| Aspergillus fumigatus | 1 |
| Candida albicans | 1 |
| Candida glabrata | 1 |
| Candida tropicalis | 1 |
| Candida parapsilosis | 1 |
| Cryptococcus victoriae or C. subarcticus | 1 |
| Endoconidioma euphorbiae | 1 |
| Malassezia restricta | 1 |
| Penicillium sp | 1 |
| Trichomonads | |
| Trichomonas spp | 2 |
| Trichomonas vaginalis | 1 |

B2. Diagnosis of T. pallidum in Positive Clinical Specimens (Accuracy Assessment)

Nine specimens were included in which either T. pallidum had been detected by 16S PCR (N=5) in clinical testing. Specifically, these specimens include Cases 36-40, of which four cases were formalin-fixed, paraffin embedded; in Cases 48 and 50, T. pallidum DNA was present below the limit of detection of the BCTDNA 16S PCR assay; in Cases 49 and 52, histopathologic staining indicated the presence of spirochetes. Case 48 was positive for T. pallidum by NGS16S only with a read-mass of 472 (0.46×lowIS) and was reported in minor abundance. DNA eluate from Case 50 was positive by tp47 FRET probe qPCR with an estimated concentration of 1.8 genomes per mcL. Case 49 was negative for bacterial DNA. Case 52 was reported as multiple templates of insufficient quantity to pursue NGS16S. Immunohistochemical staining for spirochetes was cross-reactive with other treponemes (e.g., Case 54 was positive for T. refringens, a genitourinary commensal organism) and Brachyspira spp., which were found in the anatomic site of Case 52.

The assay detected T. pallidum with melt curve Tₘ's between 84.6-84.9° C. in all replicates of Cases 36-40, 48, and 50. Cases 49 and 52 remained negative in the validation study and are thus concordant with the results from broad-range bacterial PCR. No inhibition was observed in any replicates. Full results are in Table 8.

### B3. Limit of Detection

Limit of detection was determined by performing the assay on thirty replicates of extracted T. pallidum gDNA from cultured UW228B, UW99B, and TP Nichols at concentrations between 5-20 copies per reaction, including twenty-five reactions at concentrations of 5-10 genomes per reaction (Table 10). Additionally, TP Nichols gDNA was spiked into residual negative human specimens at a concentration of 7.5 copies per reaction (1.5 copies per mcL) and was amplified by three different operators for a total of thirty replicates (see Section B4: Precision and Accuracy).

One replicate of UW228B (Table 10, Well #8) was tested negative at 10 genomes per reaction. Two replicates of UW99B (eluate E01) were tested negative at 5 genomes per reaction, but all were positive at 10 genomes per reaction. TP Nichols was consistently tested positive at 5 genomes per reaction and above. In this experiment, nineteen of twenty replicates were tested positive at concentrations of 5-10 genomes per reaction. When TP Nichols was used at 7.5 genomes per reaction, all thirty replicates were detected. In total, forty-nine of fifty replicates were positive between 7 to 10 genomes per reaction. Thus, the limit of detection is established at 7-10 genomes per reaction.

TABLE 10
Limit of Detection Replicates from 5-20 Genomes Per Reaction

| | | Copies / | Replicate 1 | | | |
|---|---|---|---|---|---|---|
| Well | Sample Name | rxn | CT | Tm1 | Date | Person/Tech |
| 1 | UW228 at 1:200k | 20 | 14.779 | 84.651 | Jul. 13, 2022 | Operator 1 |
| 2 | UW228 at 1:200k | 20 | 15.583 | 84.651 | Jul. 13, 2022 | Operator 1 |
| 3 | UW228 at 1:200k | 20 | 19.416 | 84.673 | Jul. 13, 2022 | Operator 1 |
| 4 | UW228 at 1:200k | 20 | 15.559 | 85.084 | Jul. 13, 2022 | Operator 1 |
| 5 | UW228 at 1:200k | 20 | 17.159 | 84.862 | Jul. 13, 2022 | Operator 1 |
| 6 | UW228 at 1:400k | 10 | 15.969 | 85.067 | Jul. 13, 2022 | Operator 1 |
| 7 | UW228 at 1:400k | 10 | 14.905 | 84.827 | Jul. 13, 2022 | Operator 1 |
| 8 | UW228 at 1:400k | 10 | Undetermined | 55.061 | Jul. 13, 2022 | Operator 1 |
| 9 | UW228 at 1:400k | 10 | 15.776 | 84.837 | Jul. 13, 2022 | Operator 1 |
| 10 | UW228 at 1:400k | 10 | 15.346 | 84.837 | Jul. 13, 2022 | Operator 1 |
| 11 | TP Nichols at | 10 | 9.575 | 84.661 | Jul. 1, 2022 | Operator 2 |
| | 1:200k | | | | | |
| 12 | TP Nichols at | 10 | 12.377 | 84.858 | Jul. 1, 2022 | Operator 2 |
| | 1:200k | | | | | |
| 13 | TP Nichols at | 10 | 10.492 | 84.8 | Jul. 3, 2022 | Operator 2 |
| | 1:200k | | | | | |
| 14 | TP Nichols at | 10 | 9.932 | 84.454 | Jul. 7, 2022 | Operator 1 |
| | 1:200k | | | | | |
| 15 | TP Nichols at | 10 | 10.679 | 83.804 | Jul. 7, 2022 | Operator 1 |
| | 1:200k | | | | | |
| 16 | TP Nichols at | 5 | 9.998 | 84.651 | Jul. 13, 2022 | Operator 1 |
| | 1:400k | | | | | |
| 17 | TP Nichols at | 5 | 10.003 | 84.651 | Jul. 13, 2022 | Operator 1 |
| | 1:400k | | | | | |
| 18 | TP Nichols at | 5 | 10.159 | 84.673 | Jul. 13, 2022 | Operator 1 |
| | 1:400k | | | | | |
| 19 | TP Nichols at | 5 | 10.164 | 84.879 | Jul. 13, 2022 | Operator 1 |
| | 1:400k | | | | | |
| 20 | TP Nichols at | 5 | 10.013 | 84.656 | Jul. 13, 2022 | Operator 1 |
| | 1:400k | | | | | |
| 21 | UW99B at 1:200k | 10 | 18.703 | 84.847 | Jul. 13, 2022 | Operator 1 |
| 22 | UW99B at 1:200k | 10 | 30.309 | 85.053 | Jul. 13, 2022 | Operator 1 |
| 23 | UW99B at 1:200k | 10 | 16.679 | 85.08 | Jul. 13, 2022 | Operator 1 |
| 24 | UW99B at 1:200k | 10 | 23.836 | 85.286 | Jul. 13, 2022 | Operator 1 |
| 25 | UW99B at 1:200k | 10 | 18.172 | 85.062 | Jul. 13, 2022 | Operator 1 |
| 26 | UW99B at 1:400k | 5 | 19.868 | 85.062 | Jul. 13, 2022 | Operator 1 |
| 27 | UW99B at 1:400k | 5 | 36.094 | 85.018 | Jul. 13, 2022 | Operator 1 |
| 28 | UW99B at 1:400k | 5 | 19.394 | 85.018 | Jul. 13, 2022 | Operator 1 |
| 29 | UW99B at 1:400k | 5 | Undetermined | 54.649 | Jul. 13, 2022 | Operator 1 |
| 30 | UW99B at 1:400k | 5 | Undetermined | 55.06 | Jul. 13, 2022 | Operator 1 |

### B4. Precision and Accuracy

3 mcL of TP Nichols at a concentration of 40 genomes per reaction (established by tp47 FRET qPCR in the Greninger laboratory) were spiked into 77 mcL of residual patient specimens, representing central nervous system, anorectal, oropharyngeal, ocular, and skin specimens. Specifically, Sample 2 (peri-brain fluid) contained DNA from Staphylococcus intermedius; Sample 5 (perirectal swab) contained bacterial DNA from Streptococcus anginosus, Megasphaera massiliensis, and Phocaeicola vulgatus. The other three specimens were negative by clinical testing.

Three independent operators tested all five spike-in specimens in duplicate with an associated inhibition control. Testing was performed on different days. The results were consistently reproduced across all three operators and no inhibition was observed. Positive and negative controls yielded expected results (Table 11).

The mean Tₘ across all limit-of-detection specimens and reproducibility amplifications (N=75) was 84.84° C. with a standard deviation (SD)=0.20. Similarly, the mean Tₘ across inhibition controls for experiments with Samples 1 to 70 (N=44) was 84.80° C. (SD=0.14); the mean Tₘ with gBlock controls (N=6) was 84.82° C. (SD=0.24); the mean Tₘ with gDNA from Nichols (10 copies; N=5) was 84.52° C. (SD=0.43); and the mean Tₘ with synthetic positive control (N=22) was 85.11° C. (SD=0.24).

TABLE 11
Inter-operator Reliability Study Performed at 7.5 copies of T. pallidum Nichols gDNA

| | | Jul. 13, 2022 | | Jul. 24, 2022 | | Jul. 27, 2022 | |
|---|---|---|---|---|---|---|---|
| | | Operator 1 | | Operator 2 | | Operator 3 | |
| Sample Name | Sample used | CT | Tm1 | CT | Tm1 | CT | Tm1 |
| 1: Human/TP | 220103_032 | 15.25 | 84.85 | 15.08 | 84.86 | 14.01 | 84.66 |
| Nichols | (Skin Biopsy) | | | | | | |
| 1: Human/TP | | 17.51 | 84.85 | 13.82 | 84.86 | 14.96 | 84.66 |
| Nichols | | | | | | | |
| 1: inhib | | 17.28 | 84.88 | 13.74 | 85.09 | 13.98 | 84.88 |
| 2: Human/TP | 220105_046 | 17.41 | 85.08 | 14.71 | 84.88 | 14.98 | 84.88 |
| Nichols | (Fluid Brain) | | | | | | |
| 2: Human/TP | | 19.65 | 84.86 | 17.19 | 84.87 | 18.94 | 84.87 |
| Nichols | | | | | | | |
| 2: inhib | | 16.00 | 84.86 | 14.38 | 84.87 | 15.58 | 84.87 |
| 3: Human/TP | 220201_033 | 15.14 | 84.81 | 13.75 | 85.04 | 15.75 | 84.83 |
| Nichols | (Cardiac | | | | | | |
| 3: Human/TP | Tissue) | 17.04 | 84.81 | 17.04 | 85.04 | 13.95 | 84.83 |
| Nichols | | | | | | | |
| 3: inhib | | 16.17 | 84.82 | 15.86 | 85.05 | 15.03 | 84.84 |
| 4: Human/TP | 220206_004 | 15.60 | 84.82 | 15.06 | 85.05 | 16.80 | 84.84 |
| Nichols | (eSwab | | | | | | |
| 4: Human/TP | Vaginal) | 16.53 | 84.60 | 14.00 | 84.83 | 14.74 | 84.82 |
| Nichols | | | | | | | |
| 4: inhib | | 16.28 | 84.81 | 14.60 | 84.83 | 14.85 | 84.82 |
| 5: Human/TP | 220208_005 | 15.40 | 84.85 | 14.57 | 84.66 | 14.92 | 84.45 |
| Nichols | (Perrirectal | | | | | | |
| 5: Human/TP | swab) | 17.08 | 84.85 | 15.82 | 84.66 | 16.90 | 84.66 |
| Nichols | | | | | | | |
| 5: inhib | | 16.95 | 84.88 | 15.98 | 84.88 | 15.84 | 84.88 |
| Pos | N/A | 18.02 | 84.88 | 17.90 | 85.05 | 19.99 | 85.29 |
| LC Neg | | Undetermined | 53.43 | Undetermined | 46.64 | Undetermined | 53.43 |

### B5. Evaluation of Sequencing

B5.1. Sequencing of Amplified Products from Residual Clinical Samples and gBlock Positive Control

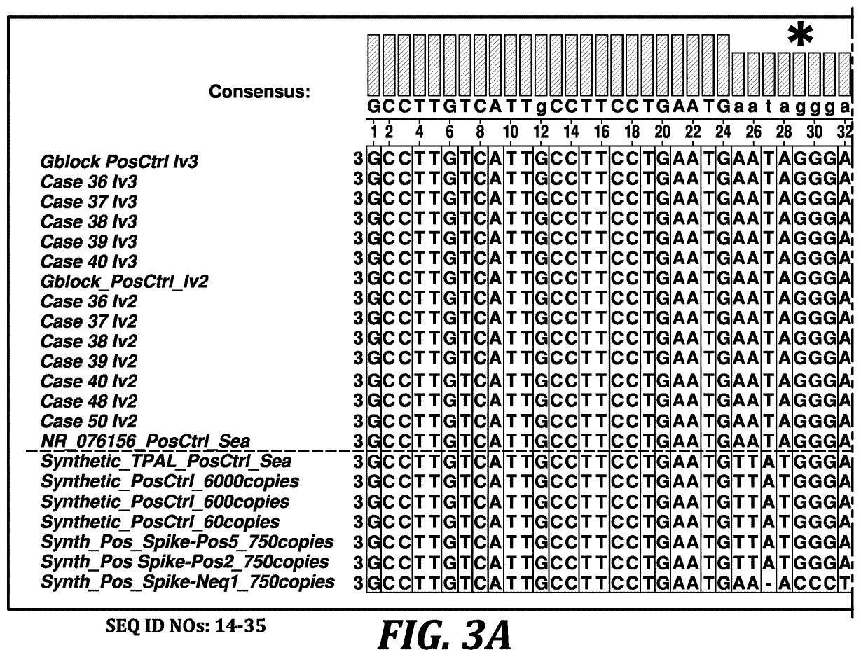

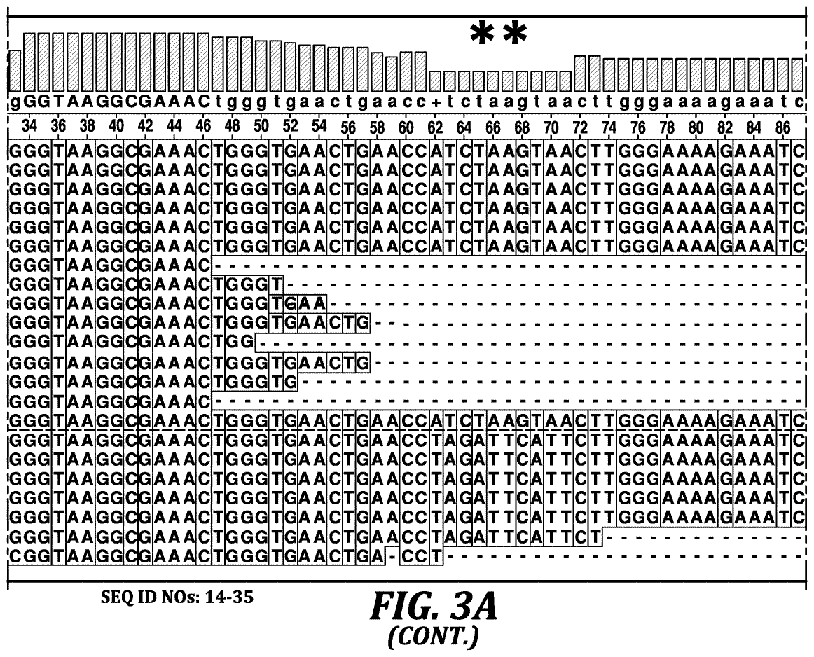

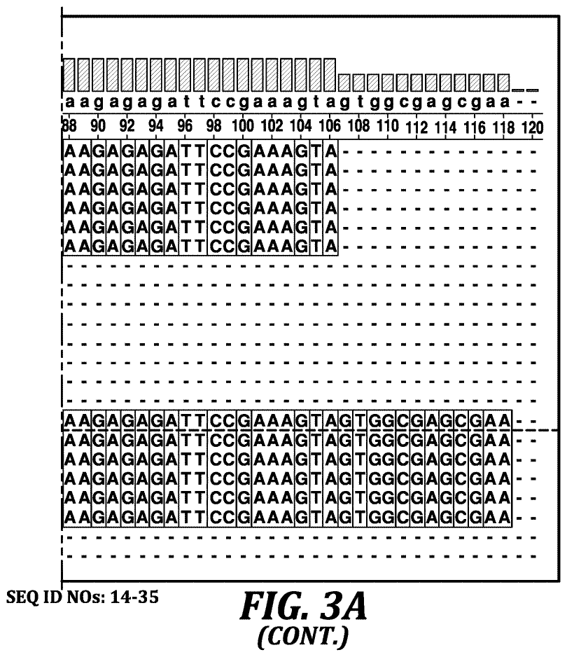

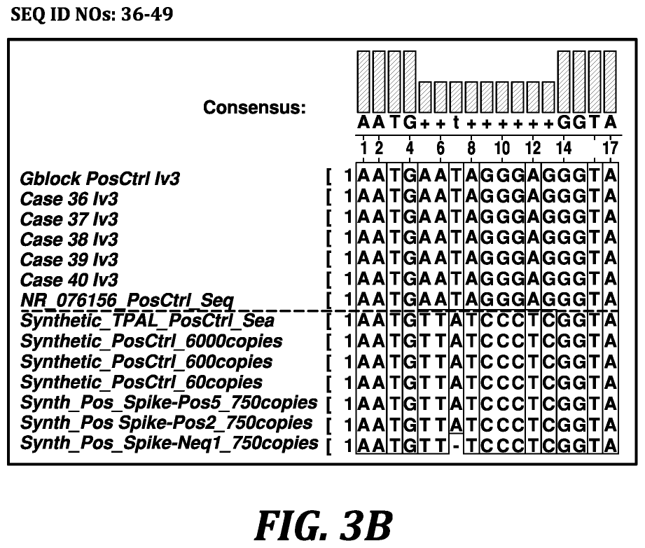

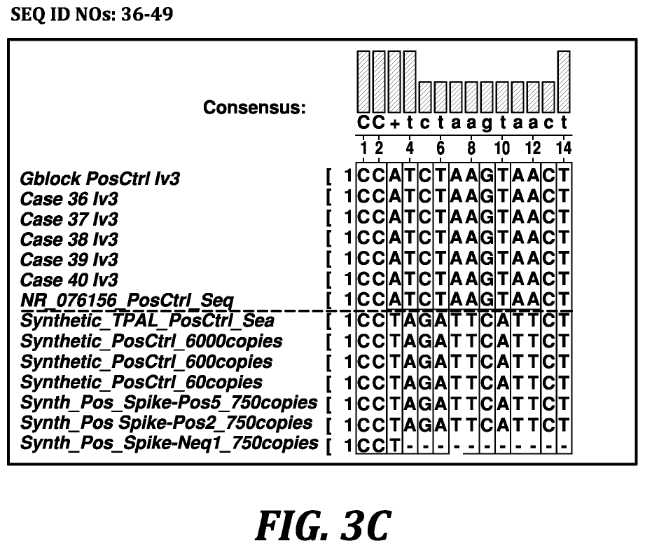

Amplified products were sequenced using current, validated procedures in the clinical laboratory (see Table 1 for primer sequences). Products from five cases and one gBlock positive control were sequenced at level 2 and level 3 dilutions. A distal portion of the reverse sequencing reaction was consistently identifiable and matched the forward strand of the reference sequence. Level 3 dilution of product consistently produced a longer, usable sequence (~110 bp) than level 2 dilution (~50 bp). The resulting sequence was consistently identified in all cases as the reference wild type T. pallidum 23S sequence (FIGS. 3A-3C).

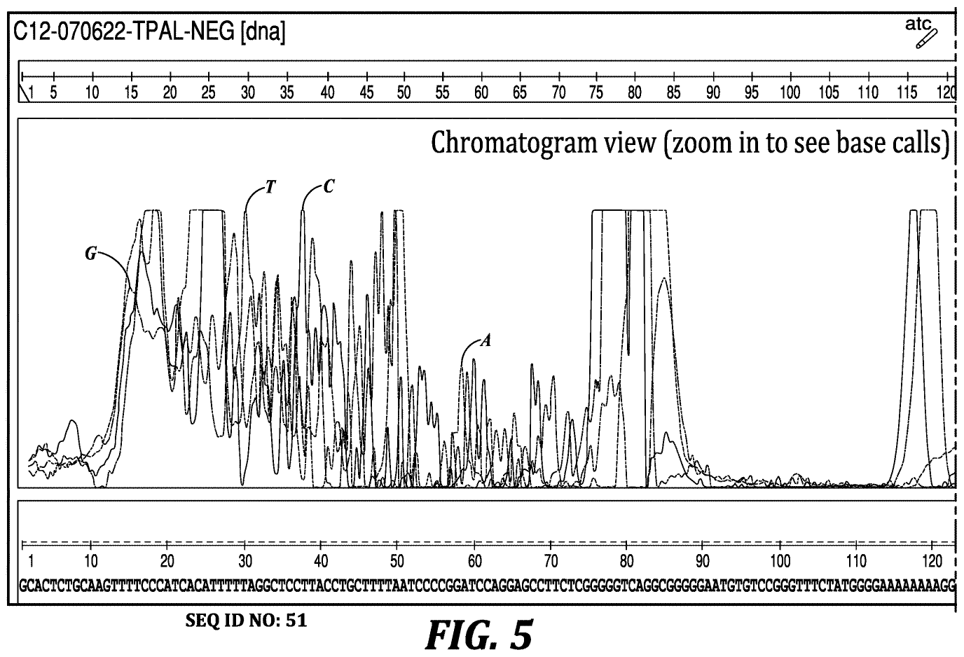

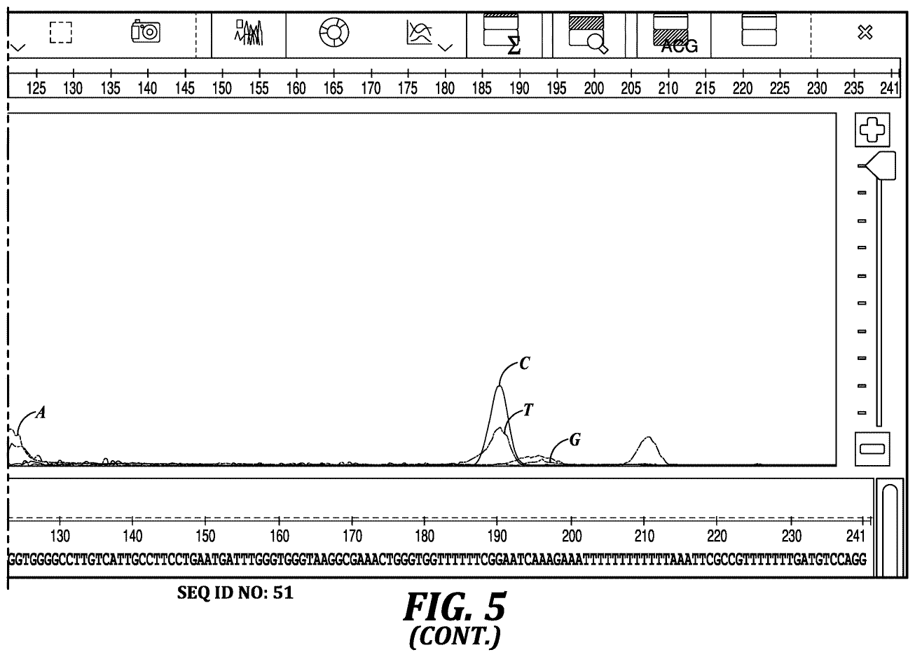

The residual clinical sample reactions that produced off-target amplifications, determined based on their non-matching Tₘ's, were also sequenced. These off-target amplified products yielded low-quality, uninterpretable sequences that did not include the reference wild type sequence (FIG. 5).

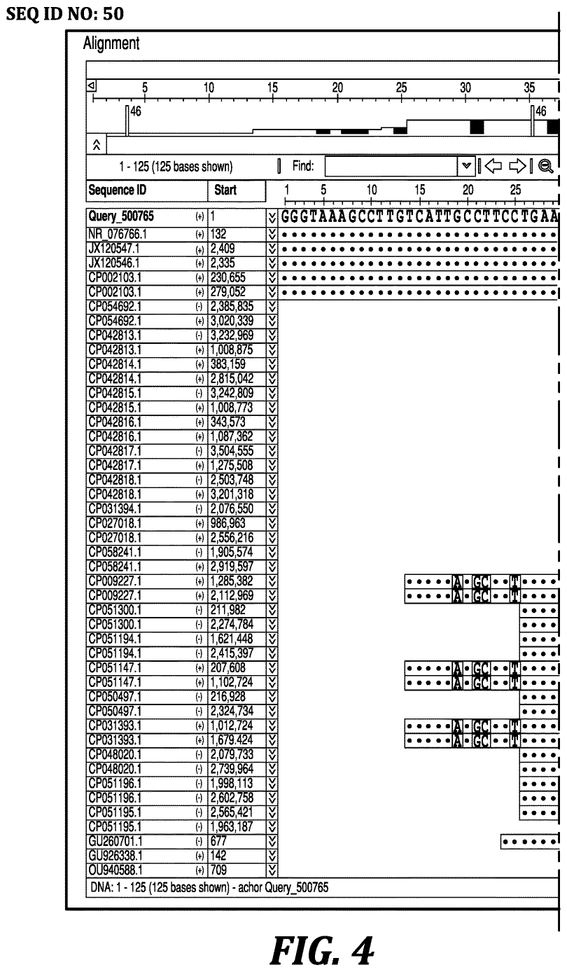

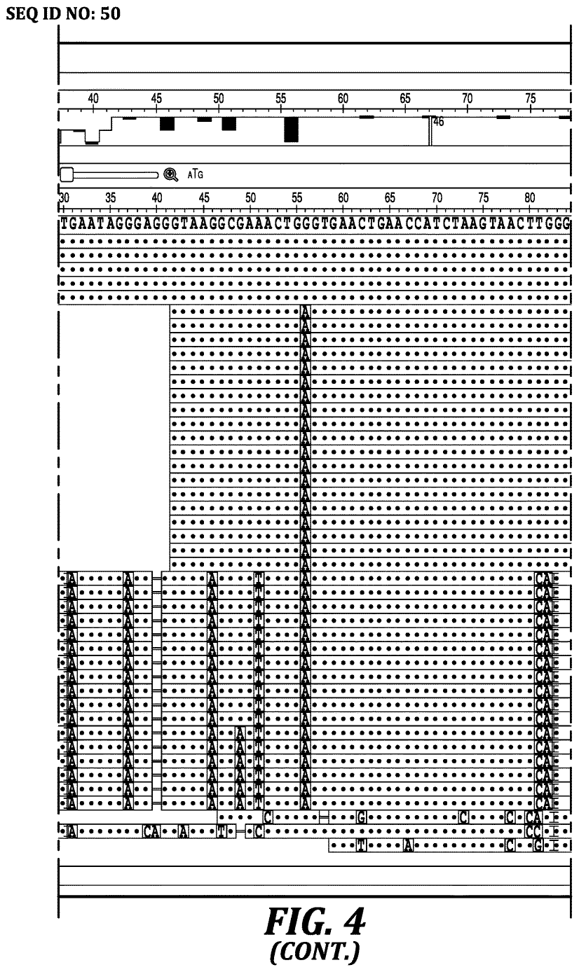

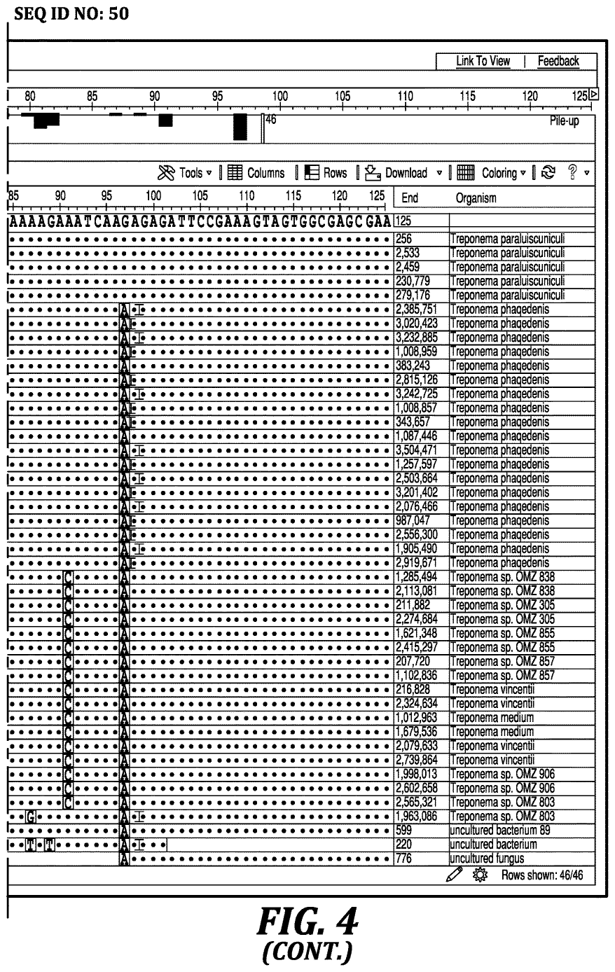

BLAST analysis of a 125 bp sequence spanning the usable sequence (the wild type version of the synthetic positive control sequence provided in Section B5.2: Sequencing of Amplified Products from Synthetic Positive Control) had 67% coverage with 96.5% identity to T. phagedenis, an animal treponeme, and 80-89% coverage with 86-96.5% identity to unnamed Treponema spp., T. vincentii, and T. medium (FIG. 4). These results indicate excellent specificity of the sequenced product for species identification. The bolded region in the wild type sequence (below) was analyzed by BLAST analysis excluding T. pallidum (taxid 160). Results indicated significant gaps in coverage and consistent polymorphisms that distinguished the T. pallidum sequence from other organisms, with the known exception of T. paraluiscuniculi, a rabbit pathogen (FIG. 4). Shown at FIG. 5, a trace file from sequencing of an NTC that produced a low-signal intensity Tₘ of 87° C. shows that sequence quality was poor, there was low signal, and the result suggests the NTC did not contain T. pallidum sequence.

B5.2. Sequencing of Amplified Products from Synthetic Positive Control

A synthetic positive control was designed with selected bases between primer-binding sites replaced by their complementary bases (see Target Sequences below for full reference and synthetic sequences). The sequence of the control was ordered from Integrated DNA Technologies (IDT) as a purified plasmid. The most relevant sequence string (below) was used to create three polymorphic stretches (bolded and in lowercase) as follows:
>Informative Sequence from Synthetic Positive Control

(SEQ ID NO: 63)
GGcatAAGCCTTGTCATTGCCTTCCTGAATGttatccctcGGTAA
GGCGAAACTGGGTGAACTGAACCtagattcattCTTGGGAAAAGA
AATCAAGAGAGATTCCGAAAGTAGTGGCGAGCGAA

Six synthetic positive control amplifications were sequenced. These six controls were either spiked into residual clinical specimens (N=3; two cases were positive, one case was negative) or mixed with pure plasmid in buffer IDTE (N=3). The resulting sequence was consistently identified as the T. pallidum synthetic positive control sequence by virtue of the introduced polymorphisms (FIG. 3).

### Target Sequences:

>NR_076156.1 Treponema pallidum subsp. pallidum strain Nichols 23S ribosomal RNA gene, Domain 1, residues 10-550

(SEQ ID NO: 13)
GTTTACGGTGGATGTCTTGGAGTTGTCAGGCGATGAAGGTCGTGA
TAAGCTGCGAAAAGCCTCGGGGAGGAGCACATGTCCTGTGATCCG
GGGATGACCGAATGGGGTAACCCGACAGGGTAAAGCCTTGTCATT
GCCTTCCTGAATGAATAGGGAGGGTAAGGCGAAACTGGGTGAACT
GAACCATCTAAGTAACTTGGGAAAAGAAATCAAGAGAGATTCCGA
AAGTAGTGGCGAGCGAAATTGGAGGAGCCTAAACCTGTGTCTAAC
AGGGGTTGTAGGGCCGCGCGGGCTTGCGTTCGGTGGGTGAAATAA
TCCGGCCTATAGCAGAAAGGTTTTGGGAAAGCCTGACAGAGAGGG
TGAAATCCCCGTATGCGGAATGGGGCGGACCTGCTGGTGCGGTAC
CTGAGTACGGCGGGACACGAGGAATCCTGTCGGAATCTGGGTCGA
CCACGATCTAAGGCTAAATACTCGACAACTACCGATAGTGGACAA
GTACC

>Synthetic TPAL Positive Ctrl

(SEQ ID NO: 64)
GTTTACGGTGGATGTCTTGGAGTTGTCAGGCGATGAAGGTCGTGA
TAAGCTGCGAAAAGCCTCGGGGAGGAGCACATGTCCTGTGATCCG
GGGATGACCGAATGGGGTAACCCGACAGGcatAAGCCTTGTCATT
GCCTTCCTGAATGttatccctcGGTAAGGCGAAACTGGGTGAACT
GAACCtagattcattCTTGGGAAAAGAAATCAAGAGAGATTCCGA
AAGTAGTGGCGAGCGAAATTGGAGGAGCCTAAACCTGTGTCTAAC
AGGGGTTGTAGGGCCGCGCGGGCTTGCGTTCGGTGGGTGAAATAA
TCCGGCCTATAGCAGAAAGGTTTTGGGAAAGCCTGACAGAGAGGG
TGAAATCCCCGTATGCGGAATGGGGCGGACCTGCTGGTGCGGTAC
CTGAGTACGGCGGGACACGAGGAATCCTGTCGGAATCTGGGTCGA
CCACGATCTAAGGCTAAATACTCGACAACTACCGATAGTGGACAA
GTACC

### B5.3. Summary of Sequencing Findings

There is 100% agreement over the sequenced region across experimentally obtained sequences and the expected positive sequence. The sequenced product from the polymorphic synthetic positive control has 100% identity with the expected sequence, but it is only consistently 82.6% identical to the unmutated control and true positive sequences over the 109 nt sequenced, reflecting the mutations designed in the control. Level 3 dilution consistently produced longer useable sequence than level 2, likely reflecting a very high concentration of LAMP product. Based on these results, it was concluded that sequencing is a reliable method to detect false-positives, including positive control carryover.

### C. Conclusions

This example tested the performance of a laboratory-developed LAMP assay for detection of T. pallidum in clinical specimens by amplifying the 23S IRNA gene. The performance of the assay was tested using 1) extracted gDNA from cultured T. pallidum clinical and laboratory isolates, 2) a variety of residual clinical specimens with closely related treponemes and more distantly related, clinically relevant organisms, and human DNA, and 3) synthetic dsDNA controls representing the homologous 23S rRNA sequences of closely related treponemes and uncultivable and/or uncommon organisms found in anatomically relevant sites. The analytical sensitivity (95% limit of detection) was determined to be 7-10 genomes per reaction. Using melt curve analysis alone, the assay consistently identified all expected positives above the limit of detection (100% accuracy), and all specificity controls were expectedly negative (100% analytical specificity). Melt curve analysis also distinguished true positives from off-target amplification; such off-target events were rare (<5%), always had significantly different melt curve profiles, and were consistently low in fluorescence intensity. Precision was determined by three different clinical operators amplifying T. pallidum gDNA spiked into five residual clinical specimens at 7.5 genomes per reaction (N=10 reactions per operator) on three different days; no inter-operator variation was observed. The reference interval is “not detected” and the reportable range is not applicable to this test with a binary outcome. A synthetic positive control was also developed. Sequencing was demonstrated as a reliable method to discriminate between true positives, control carryover, and off-target amplification. Based on the above findings, the performance of this LAMP assay for T. pallidum DNA detection (TPLDNA: Treponema pallidum DNA detection) is considered acceptable for patient testing, and this assay represents a novel adjunct in syphilis diagnosis.

### Example 2

Treponema pallidum Detection by Loop-Mediated Isothermal Amplification Validation in Anticoagulated Whole Blood

In addition to Example 1, which describes in detail the LAMP assay for Treponema pallidum (T. pallidum) detection using direct patient non-blood specimens, this example describes further work validating the same assay using matrix specimens, in order to meet significant clinical demand. Specifically, in this example, EDTA-anticoagulated whole blood (lavender or purple top phlebotomy tubes) was tested. The molecular microbiology laboratory extracts total nucleic acids from blood using both the EZ2 instrument and the EasyMAG instrument. Since reverse transcriptase is incorporated into the assay reagents, this method can capitalize on the high transcript copies of rRNA as a pre-analytical signal amplification.

Estimated sensitivities of molecular syphilis assays performed in peripheral blood specimens range from 14-54% for primary syphilis and 58-82% for secondary syphilis. Whole blood and peripheral blood mononuclear cells appear to represent a useful specimen type. Specifically, a DNA LAMP assay of the disclosure has a reported clinical sensitivity of 82% in anticoagulated blood drawn from a cohort of patients with high pre-test probability of secondary syphilis. Using serum, a highly-sensitive RT-TMA reported sensitivities of 37.5% in primary syphilis and 35.7% in secondary syphilis. These findings make whole blood a logical specimen target for TPLDNA.

The validation described in this example relied on blood from lavender top tubes spiked with known concentrations of cultured T. pallidum and quantified by darkfield microscopy counts to determine the limit of detection (analytical sensitivity), accuracy, and precision/inter-operator reliability. This example included randomly-selected residual lavender top tubes and residual specimens known to be positive for viruses that are frequently found as concomitant infections in syphilis patients or whose viral lesions mimic those of syphilis. Additionally, total nucleic acid eluates positive for monkeypox virus and extracted in the virology laboratory are also tested and compared to syphilis serologic status of those patients. Thus, this example extends the specificity study performed in Example 1.

### A. Methods

A1. Primer Design and In silico Analysis

Refer to Example 1: Section A1 (Primer Design) and A2 (In silico Analysis of 23S IRNA Sequences from Closely and Distantly Related Organisms).

### A2. Reaction Conditions

Reaction conditions in this example were identical to those described in Example 1: Section A.3 (Reaction Conditions).

### A3. Strains and Tested Specimens

### A3.1 Preparation of Positive Specimens

Cultured isolates of T. pallidum strains SS14, Chicago C, and Nichols were cultivated as described. Organisms were quantified by darkfield microscopy and inoculated into uninfected EDTA-anticoagulated whole blood (WB) at decreasing concentrations of ~1×10⁶, 1×10⁵, 1×10⁴, 1×10³, 0.5×10³, and 0.25×10³ organisms (CFU) per mL for each strain (spike-in specimens). A second set of T. pallidum cultures (strains as above) were harvested, quantified by darkfield microscopy organism counts, and inoculated as follows: strain Nichols into a single random residual WB; SS14 and Chicago (separately) into a pool of 4 residual WB specimens. All treponemes were spiked at 1000 organisms/mL.

### A3.2 Selection of Specificity Controls

Four randomly selected WB specimens were included. One of each of the following specimens were also included: 1) residual serum positive for Varicella Zoster Virus (VZV), 2) residual plasma positive for Herpes Simplex Virus 1 (HSV-1), Herpes Simplex Virus 2 (HSV-2), Human Immunodeficiency Virus (HIV)-1, Hepatitis B Virus (HBV), and Hepatitis C Virus (HCV), and 3) Monkeypox Virus (MPX) from a clinical specimen. Four of these viruses produce lesions for which syphilis is in the differential—VZV, HSV1, HSV2, and MPX—while HCV, HBV, and HIV can be detected in blood. Additionally, studies have reported coinfection of MPX and syphilis: >6% in some studies. Similarly, populations at risk of syphilis also have risk factors for HIV and MPX infection.

### A4. Nucleic Acid Extraction

All specimens were processed following clinically validated protocols in the CLIA laboratory. Aliquots of spike-in specimens were extracted separately by both EasyMAG and EZ2 (Qiagen). Specificity controls were extracted by EZ2. The MPX nucleic acid was extracted by MP96. Aliquots of spike-in specimens were extracted separately by both EasyMAG and EZ2 (Qiagen) for the first set of spike-ins; for the second set of spike-ins, only EZ2 was used. Specificity controls were extracted by EZ2. The MPX nucleic acid was extracted by MP96 using a clinically validated method by the virology clinical laboratory.

### A4.1 Estimation of Genome Copies in Extracted Nuclei Acid Eluates

Predicted genomes represented per reaction based on darkfield microscopy are calculated in Table 12. Nucleic acid from the highest two concentration spike-in eluates from the initial experiment (1×10⁶ and 1×10⁵ cfu/mL) were interrogated by a quantitative polymerase chain reaction (qPCR) for the single-copy gene tp47 and compared to a standard curve to estimate target copy number in the extracted samples. For the second experiment, involving both a pool of 4 and an individual residual WB with T. pallidum spiked-in at 1×10³ organisms/mL, eluates were not interrogated by tp47 qPCR since analyte concentration was expected to be below the LOD of the qPCR assay.

TABLE 12
Calculated Genomes per Reaction by Darkfield Microscopy Counts

| Extraction Method | EZ2 | EasyMAG |
|---|---|---|
| Initial Inoculum (organisms / mL) | 1000 | 1000 |
| Initial Inoculum (organisms / mcL) | 1 | 1 |
| Volume of WB (in mcL) | 100 | 50 |
| Fold Dilution in Lysis Buffer (2 mL) | 21 | 41 |
| Lysis Buffer + Sample Loaded (mcL) | 400 | 900 |
| Organisms per Extraction | 19 | 22 |
| Eluate Volume (40 mcL) | 40 | 60 |
| Expected genomes per mcL Eluate | 0.48 | 0.37 |
| Expected genomes per 5 mcL Rxn | 2.4 | 1.8 |

### B. Results

### B1. Analytical Sensitivity

Spike-in specimens were tested in triplicate and with an inhibition control (half template volume, half synthetic positive control) by three different operators on two different days, including different shifts on the second day (Table 13). T. pallidum was detected in all replicates of the two highest spike-in concentrations (1×10⁶ and 1×10⁵ CFU per mL; N=6 each, SS14 only; data not shown) and in 100% of eighteen replicates from spike-in controls with all three strains at 1,000 CFU per mL (Table 14). T. pallidum was detected in 72% and 56% of extracted nucleic acid from 500 CFU per mL and 250 CFU per mL spike-in controls, respectively (Table 14). No inhibition was observed.

For the second experiment, spike-in specimens were tested by 2 different operators on 2 different days. Assays were performed in technical duplicate plus inhibition controls, on-plate negative controls (NTC), and an on-plate positive control. T. pallidum was detected in all samples (Table 13 and Table 14). Again, no inhibition was seen, and all controls were appropriately positive or negative.

Amplifications from the first set of spike-in samples demonstrated CT values of 13-15 at inoculum concentrations of 1000 organisms/mL of WB. In contrast, all specimens in the second set of spike-in experiments had much higher Ct's (all >35, save for one replicate of Chicago in the pooled WB), suggesting in this experiment darkfield overcounted treponemes in the specimen.

The concentration of T. pallidum genomes was also estimated by amplifying tp47. Results indicated 100% amplification in replicates with a concentration between ~10-15 copies per reaction, 89% amplification in replicates with 5-10 copies per reaction, and 76% amplification with 5 or fewer copies per reaction (Table 14). Altogether, the results suggest a limit of detection of ~10 copies per reaction in blood or approximately 1,000 CFU per mL as quantified by darkfield microscopy.

TABLE 13
Reproducibility of T. pallidum in Whole Blood, Concentration
Estimated by CFU Count Using Spike-in Controls

| | Reaction (CFU/mL) | | | | | |
|---|---|---|---|---|---|---|
| Median Est. Copies | 1.5-3 CFU | | 0.8-1.4 CFU | 0.4-0.7 CFU | | |
| SS14 | 6 | 6 | 1 | 6 | 2 | 6 |
| Chicago | 6 | 6 | 6 | 6 | 3 | 6 |
| Nichols | 6 | 6 | 6 | 6 | 5 | 6 |
| Totals | 18 | 18 | 13 | 18 | 10 | 18 |
| % Positive | 100% | 100% | 72% | 100% | 56% | 100% |

TABLE 14
Reproducibility of T. pallidum in Whole Blood,
Concentration Estimated by CFU Count Using tp47

| | Reaction (tp47) | | | | | |
|---|---|---|---|---|---|---|
| Median Est. | >10 | | 5.1-10 | | ≤5 | |
| Copies | positive | total | positive | total | positive | total |
| SS14 | 3 | 3 | 4 | 6 | 7 | 9 |
| Chicago | 6 | 6 | 6 | 6 | 4 | 6 |
| Nichols | 6 | 6 | 6 | 6 | 5 | 6 |
| Totals | 15 | 15 | 16 | 18 | 16 | 21 |
| % Positive | 100% | 100% | 89% | 100% | 76% | 100% |

TABLE 15
Detailed Experimental Results

| | Sample Information | | | | |
|---|---|---|---|---|---|
| | | T pallidum | | | |
| | | Strain or | | | |
| Sample | | Specificity | Sample | | |
| Ref. | Dates | Target | Type | Inoculum | Amp |
| 1 | Aug. 16, 2022 | SS14 | EDTA-WB | 1.00E+05 | TPLDNA |
| 2 | Aug. 16, 2022 | SS14 | EDTA-WB | 1.00E+04 | TPLDNA |
| 3 | Aug. 18, 2022 | Chicago C | EDTA-WB | 1.00E+04 | TPLDNA |
| 4 | Aug. 16, 2022 | SS14 | EDTA-WB | 1.00E+03 | TPLDNA |
| 5 | Aug. 18, 2022 | Chicago C | EDTA-WB | 1.00E+03 | TPLDNA |
| 6 | Aug. 18, 2022 | Nichols | EDTA-WB | 1.00E+03 | TPLDNA |
| 7 | Aug. 16, 2022 | SS14 | EDTA-WB | 500 | TPLDNA |
| 8 | Aug. 18, 2022 | Chicago C | EDTA-WB | 500 | TPLDNA |
| 9 | Aug. 18, 2022 | Nichols | EDTA-WB | 500 | TPLDNA |
| 10 | Aug. 16, 2022 | SS14 | EDTA-WB | 250 | TPLDNA |
| 11 | Aug. 18, 2022 | Chicago C | EDTA-WB | 250 | TPLDNA |
| 12 | Aug. 18, 2022 | Nichols | EDTA-WB | 250 | TPLDNA |
| 13 | 8/16, 8/18 | NTC 1 | EDTA-WB | 0 | TPLDNA |
| 14 | 8/16, 8/18 | NTC 2 | EDTA-WB | 0 | TPLDNA |
| 15 | 8/16, 8/18, 8/29 | Neg Ctrl 1 | EDTA-WB | 0 | TPLDNA |
| | | (random) | (r1) | | |
| 16 | 8/16, 8/18, 8/29 | Neg Ctrl 2 | EDTA-WB | 0 | TPLDNA |
| | | (random) | (r2) | | |
| 17 | 8/16, 8/18, 8/29 | Neg Ctrl 3 | EDTA-WB | 0 | TPLDNA |
| | | (random) | (r3) | | |
| 18 | 8/16, 8/18, 8/29 | Neg Ctrl 4 | EDTA-WB | 0 | TPLDNA |
| | | (random) | (r4) | | |
| 19 | Sep. 20, 2022 | SS14 | EDTA-WB | 1.00E+03 | TPLDNA |
| | | | (pool) | | |
| 20 | Sep. 22, 2022 | Chicago C | EDTA-WB | 1.00E+03 | TPLDNA |
| | | | (pool) | | |
| 21 | 9/20 & 9/22 | Nichols | EDTA-WB | 1.00E+03 | TPLDNA |
| | | | (r5) | | |
| 22 | Aug. 29, 2022 | HSV1 | plasma | N/A | TPLDNA |
| 23 | Aug. 29, 2022 | HSV2 | plasma | N/A | TPLDNA |
| 24 | Aug. 29, 2022 | VZV | serum | N/A | TPLDNA |
| 25 | Aug. 29, 2022 | MPX | swab | Ct < 25 | TPLDNA |
| | | (diluted 1:5) | | | |
| 26 | Aug. 29, 2022 | HIV-1 | plasma | N/A | TPLDNA |
| 27 | Aug. 29, 2022 | HBV | plasma | N/A | TPLDNA |
| 28 | Aug. 29, 2022 | HCV | plasma | N/A | TPLDNA |

| | EasyMAG | | |
|---|---|---|---|
| | | | N (%) |
| Sample Ref. | Ct* | Tm* | Positive |
| 1 | 10.037 | 85.056 | 3 (100) |
| 2 | 11.966 | 85.176 | 3 (100) |
| 3 | 11.878 | 84.718 | 3 (100) |
| 4 | 15.181 | 85.155 | 3 (100) |
| 5 | 13.234 | 84.843 | 3 (100) |
| 6 | 13.021 | 84.304 | 3 (100) |
| 7 | 27.116 | 85.253 | 1 (33.3) |
| 8 | 14.368 | 84.754 | 3 (100) |
| 9 | 12.837 | 84.431 | 3 (100) |
| 10 | N/A | N/A | 0 (0) |
| 11 | 22.489 | 84.547 | 2 (66.67) |
| 12 | 15.914 | 0.722 | 2 (66.67) |
| 13 | N/A | 53.49 | 0 (3) |
| 14 | N/A | 54.65 | 0 (3) |
| 15 | 19.236 | 54.5535 | 0 (2) |
| 16 | 10.084 | 53.426 | 0 (2) |
| 17 | N/A | 54.234 | 0 (2) |
| 18 | N/A | 53.8275 | 0 (2) |
| 19 | | | |
| 20 | | | |
| 21 | | | |
| 22 | | | |
| 23 | | | |
| 24 | | | |
| 25 | | | |
| 26 | | | |
| 27 | | | |
| 28 | | | |

| Totals at 1e3 organisms / mL | 9 (100%) |
|---|---|
| Totals | 29 (80.6) |

| | EZ2 | | |
|---|---|---|---|
| | | | N (%) |
| Sample Ref. | Ct* | Tm* | Positive |
| 1 | 11.21 | 84.576 | 3 (100) |
| 2 | 14.008 | 84.628 | 3 (100) |
| 3 | 12.993 | 84.239 | 3 (100) |
| 4 | 17.169 | 84.608 | 3 (100) |
| 5 | 16.974 | 84.363 | 3 (100) |
| 6 | 17.238 | 84.099 | 3 (100) |
| 7 | N/A | N/A | 0 (0) |
| 8 | 15.654 | 84.206 | 3 (100) |
| 9 | 15.962 | 84.02 | 3 (100) |
| 10 | 18.417 | 84.648 | 2 (66.67) |
| 11 | N/A | 85.78 | 2 (66.67) |
| 12 | 22.046 | 84.062 | 3 (100) |
| 13 | N/A | 54.517 | 0 (3) |
| 14 | N/A | 54.376 | 0 (3) |
| 15 | | | |
| 16 | | | |
| 17 | | | |
| 18 | | | |
| 19 | 34.02 | 84.53 | 2 (100%) |
| 20 | 39.93 | 84.14 | 2 (100%) |
| 21 | 37.52 | 84.09 | 4 (100%) |
| 22 | 36.95 | 87.73 | 0 (0) |
| 23 | 36.91 | 87.54 | 0 (0) |
| 24 | 32.00 | 87.68 | 0 (0) |
| 25 | 31.83 | 87.12 | 0 (0) |
| 26 | 39.23 | 86.52 | 0 (0) |
| 27 | N/A | N/A | 0 (0) |
| 28 | N/A | N/A | 0 (0) |
| | Totals at 1e3 organisms / mL | | 17 (100%) |
| | Totals | | 39 (88.6) |

*Average values across technical replicates; replicates that did not amplify were dropped from calculation.

### B2. Reproducibility

The sensitivity experiments performed above were readily reproduced by three independent operators. The assay was therefore concluded to be reproducible.

### B3. Analytical Specificity

No amplification was observed in any of the seven specificity controls or the four negative whole blood specimens (each tested in technical duplicate with an inhibition control reaction). Sporadic, late-amplifying signal was detected in four of twenty-two reactions (one technical replicate for each of HCV, MPX, VZV, and HSV-2), but it was readily distinguished from true positives by melt curve analysis, which demonstrated a difference in Tₘ greater than or equal to 2° C. from positive and inhibition controls and thus scored as NOT DETECTED (Table 15). No inhibition was observed. Coupled with the specificity analysis performed in Example 1, the assay was concluded to remain specific in blood, and these common viruses do not constitute interferences.

### B4. Sequencing

Three representative amplifications from low template concentration LAMP reactions were sequenced as described in the TPLDNA master validation (FIG. 2). Each of these produced the expected wild type sequence, ruling out splash-over contamination from the synthetic positive control and matching the wild type sequence with 100% identity. Both polymorphic regions were covered.

### C. Conclusions

This addendum validation study extends the conclusions of the primary validation to EDTA anticoagulated whole blood (lavender top tubes). Analytical Sensitivity is consistent with the primary results: the assay can reliably detect ~10 copies of target nucleic acid when extracted from blood, corresponding to approximately ~1000-5000 T. pallidum organisms per milliliter. Analytical Specificity remains at 100% as blood does not cause assay inhibition and other viruses which commonly co-occur in T. pallidum infections and/or are clinical mimics of syphilitic lesions do not interfere with the assay/were readily scored as negative. Precision was confirmed by successful and robust assay performance across multiple operators on different days and shifts. Accuracy remains 100% as all expected samples at or above the LOD were detected and sequencing remains capable of distinguishing synthetic positive control sequences from wild type (clinical/true positive) sequences. Whole blood from known clinical true positives was not available and was not assessed. The reference interval (“not detected”) and reportable range (not applicable) are unchanged from primary validation.

Based on the above findings, together with the results from the primary validation, the performance of this method (TPLDNA: Treponema pallidum DNA detection) is considered acceptable for patient testing.

### Example 3

### LAMP Primer Comparison

This example shows the comparison of the two candidate primer sets (Integrated DNA Technologies) used in the LAMP assay described in Examples 1 and 2 (Table 16). These primer sets were selected based on bioinformatic analysis and their performance in experiments assessing sigmoidal increase in fluorescence and cycle threshold (C_{T}) value. Such assessments used genomic DNA (gDNA) extracted from cultures of Treponema pallidum (TP) Nichols and two clinical isolates as template.

Five LAMP primer sets were designed using the New England Biolabs LAMP primer design tool <https://lamp.neb.com/#!/>. The primers were designed to target the proximal 23S rRNA gene V1 domain, a region containing polymorphisms that distinguish TP from non-TP treponemal species. Two sets, P3L2 and PIL1 (Table 14), maximally overlaid sequence polymorphisms differentiating TP from other mucosal microbiota, including non-TP Treponema spp., such as T. denticola.

These primer sets were evaluated using a synthetic fragment of the TP 23S gene as a positive control, as well as extracted gDNA from four cultured TP isolates, including laboratory strains Nichols and SS14, and two clinical isolates (UW 091B and UW 0228B). Five to 100 copies of TP gDNA, quantified by tp47 qPCR, were spiked into 55 ng of human DNA background to simulate clinical testing matrices.

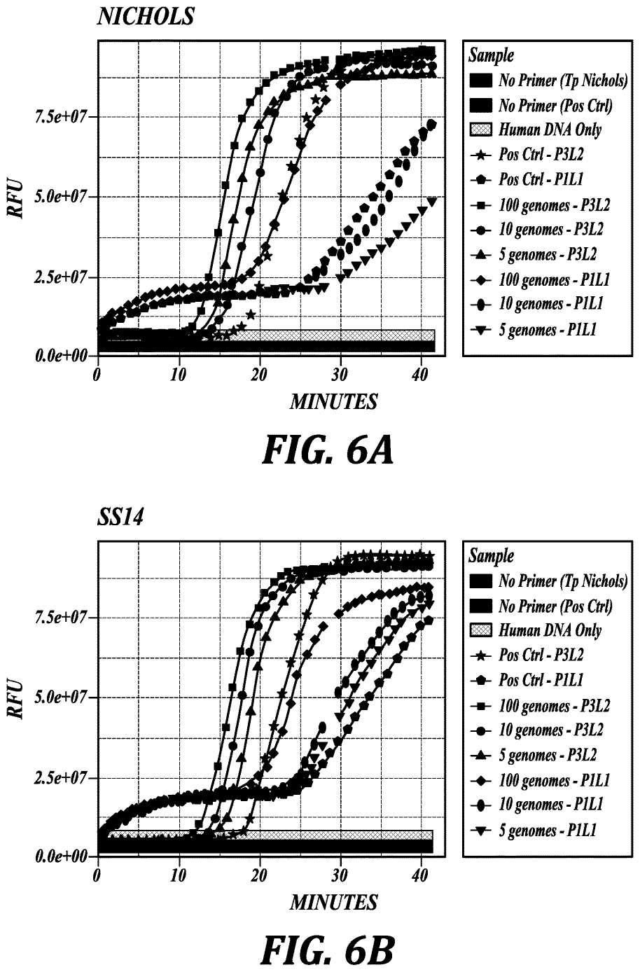

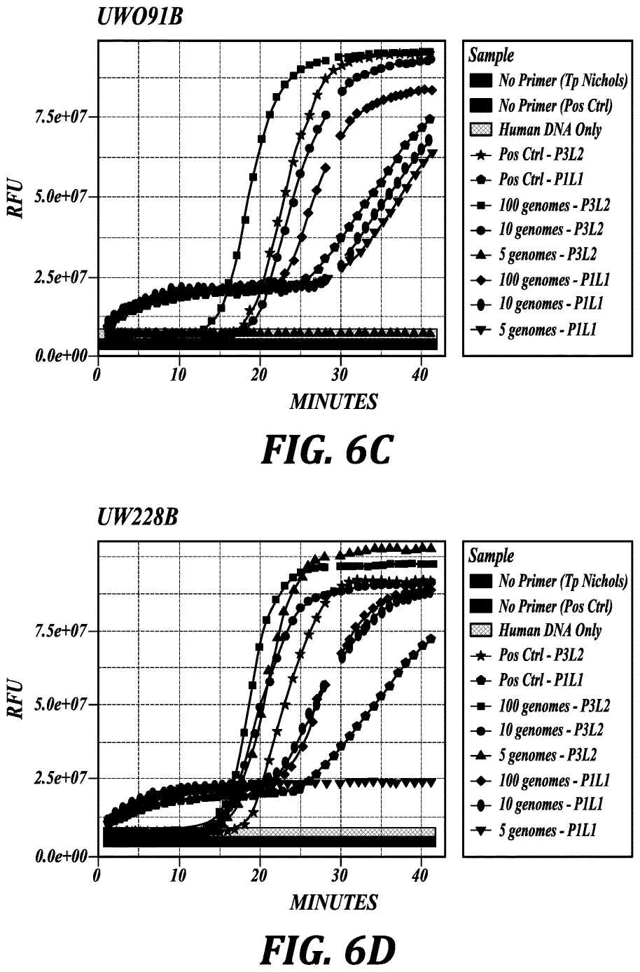

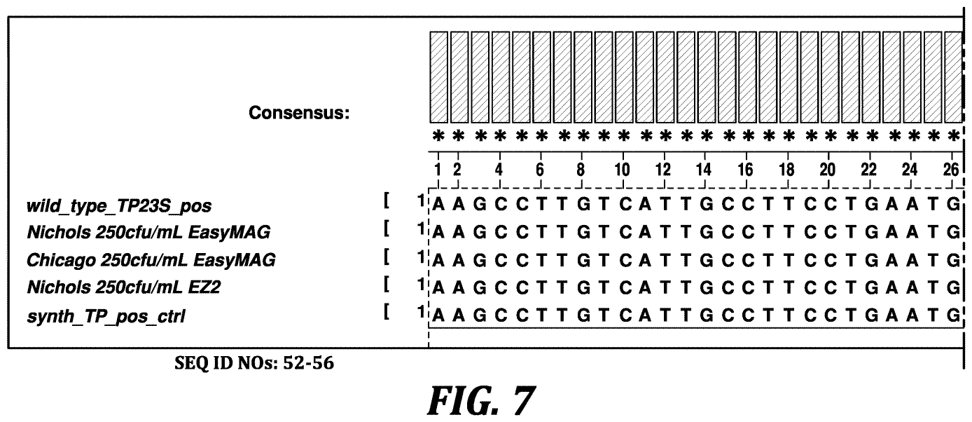

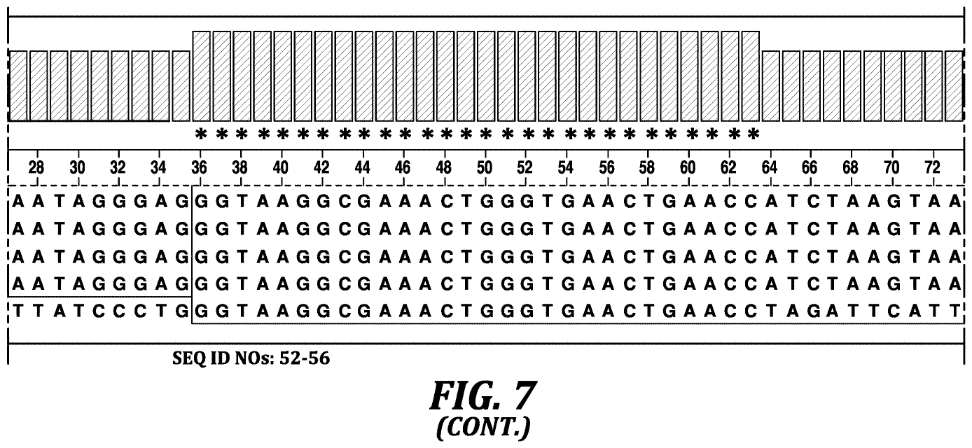

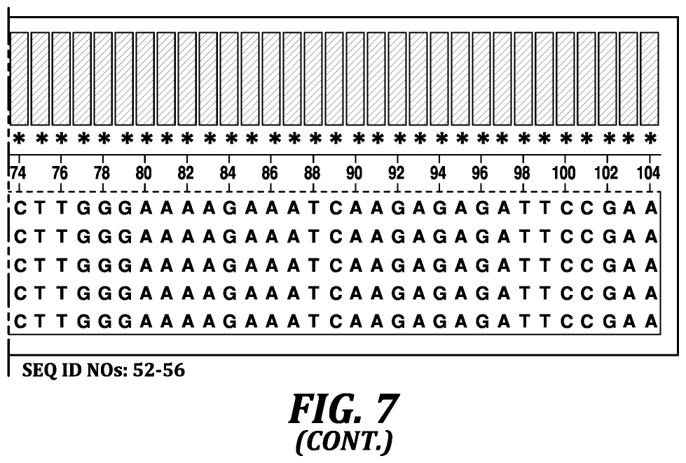

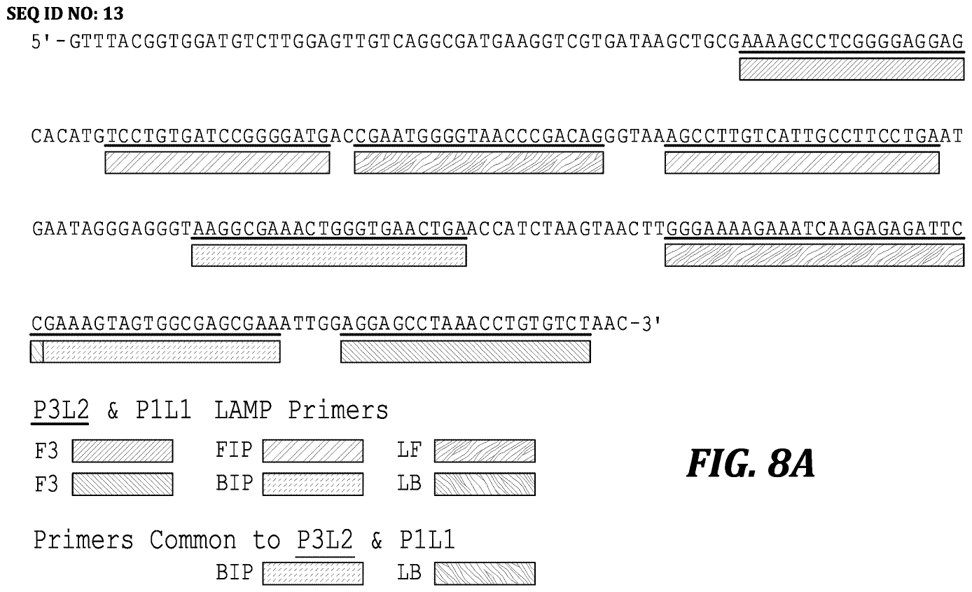

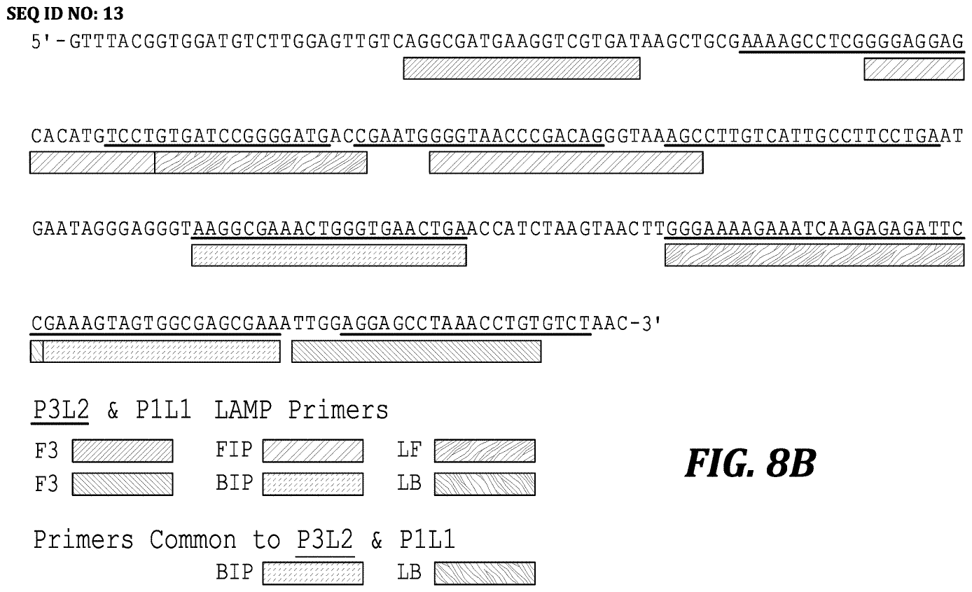

Primer set P3L2 consistently showed amplification across all concentrations of gDNA with a lower time to amplification (assessed based on C_{T}) than PIL1 (FIGS. 6A-6D). Additionally, the amplification curve was more consistently sigmoidal in accordance with the expected kinetics of nucleic acid amplification (FIGS. 6A-6D). Given these observations of primer performance, the P3L2 primer set was selected for validation of the LAMP assay described in Examples 1 and 2.

TABLE 16
Primer Sequences Tested in Syphilis LAMP Assay Development

| Primer | Primer | | Scale | Purific |
|---|---|---|---|---|
| Set | Name | Sequence | (nm) | ation |
| P3L2 | F3 | AAAAGCCTCGGGGAGGAG (SEQ ID NO: 1) | 25 | STD |
| | B3 | AGACACAGGTTTAGGCTCCT (SEQ ID NO: 2) | 25 | STD |
| | FIP | TCAGGAAGGCAATGACAAGGCTTCCTGTG | 100 | STD |
| | | ATCCGGGGATG (SEQ ID NO: 3) | | |
| | BIP | AAGGCGAAACTGGGTGAACTGATTCGCTC | 100 | STD |
| | | GCCACTACTTTC (SEQ ID NO: 4) | | |
| | LF | CTGTCGGGTTACCCCATTCG (SEQ ID NO: 5) | 25 | STD |
| | LB | GGGAAAAGAAATCAAGAGAGATTCC (SEQ | 25 | STD |
| | | ID NO: 6) | | |
| PIL1 | F3 | AGGCGATGAAGGTCGTGAT (SEQ ID NO: 7) | 25 | STD |
| | B3 | ACAGGTTTAGGCTCCTCCAA (SEQ ID NO: 8) | 25 | STD |
| | FIP | GCTTTACCCTGTCGGGTTACCCGGGAGGAG | 100 | STD |
| | | CACATGTCCT (SEQ ID NO: 9) | | |
| | BIP | AAGGCGAAACTGGGTGAACTGATTCGCTC | 100 | STD |
| | | GCCACTACTTTC (SEQ ID NO: 10) | | |
| | LF | GGTCATCCCCGGATCAC (SEQ ID NO: 11) | 25 | STD |
| | LB | GGGAAAAGAAATCAAGAGAGATTCC (SEQ | 25 | STD |
| | | ID NO: 12) | | |

FIGS. 6A-6D show LAMP Amplification Curves of Genomic DNA from Four T. pallidum Strains. Briefly, extracted DNA was diluted to 100 (squares), 10 (circles), and 5 (triangles) genome copies per reaction and amplified with LAMP primer set P3L2 or PIL1. Positive control reactions were performed with 360 copies per reaction of synthetic DNA template. Negative control reactions lacking primer are shown in black, and human DNA-only control amplifications are shown in grey.

### Example 4

Clinical Validation of a Loop-Mediated Isothermal Amplification (LAMP) for Detection of Treponema pallidum

This example describes the clinical validation of the LAMP assay for detection of T. pallidum, the etiologic agent for syphilis. As shown in Examples 1 and 2, this assay detects T. pallidum by targeting and amplifying the 23S rRNA of the bacterium using the LAMP reaction. The performance characteristics have been established based on analytical sensitivity and specificity, precision, and accuracy using a panel of genomic DNA (gDNA) extracted from cultured clinical isolates, residual clinical specimens, and synthetic DNA controls (Examples 1 and 2).

Amplifications of T. pallidum gDNA demonstrated a 95% limit of detection (LOD) of 7-10 genomes per reaction. Specificity experiments were performed with twenty-nine synthetic 23S rRNA gene sequences, representing nineteen Treponema spp, and ten clinically relevant bacteria; six specimens containing only human gDNA; and forty-six residual clinical samples that had tested positive for non-T. pallidum organisms. Residual clinical samples included 114 identified non-TP bacterial species, eight fungal species, and three trichomonads identified as or related to T. vaginalis. Bacteria included six non-pallidum treponemes, four members of Family Treponemataceae, and one member of Order Spirochaetales. Off-target amplification was <5% (12/281 reactions) and readily distinguishable from TP by either melt curve or sequence analysis. Accuracy was established by demonstrating successful amplification in seven cases positive for T. pallidum, two of which could only be detected by clinical amplicon next-generation sequencing or research-only qPCR. Reproducibility was established by amplification of T. pallidum Nichols gDNA in residual clinical DNA eluates (7.5 genomes per reaction) by three operators on three different days. Inhibition and non-template controls were performed in parallel; inhibition was not observed. Assessment of all results indicated that this assay is effective and available for patient care with a turnaround time of 48-60 hours.

### Example 5

### Summary of Clinical Testing Results

The disclosed LAMP assay was used to test for the presence of syphilis in clinical samples from human subjects, and results are shown at Table 17 and Table 18.

TABLE 17
Clinical testing results

| | Number (%) | |
|---|---|---|
| Tests Performed | Positive | Notes |
| Total Tests | 110 (100) | |
| All Positive Tests | 7 (6.6) | 6 / 7 FFPE* |
| Primary Syphilis | 2 (1.8) | Probable primary disease |
| Secondary Syphilis | 5 (4.5) | Includes two rare anatomic |
| | | sites, stomach & aorta |

*FFPE = formalin-fixed paraffin-embedded tissue

TABLE 18
Clinical testing results

| Negative Test Results | Number |
|---|---|
| Negative Tests | 103* |
| Alternative Pathogen | 13 |
| Negative Serology | 2 |
| Stain/Histology Negative | 12 |

*Testing identified at least three false-positive/cross-reactive immunohistochemical stains in histopathology.

In additional view of the successful clinical tests, it was concluded that this assay is effective and available for patient care with a turnaround time of 48-60 hours.

The complete disclosure of all patents, patent applications, and publications, and electronically available material cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

As used herein, the term “positive,” when referring to a result or signal, indicates the presence of an analyte or item that is being detected in a sample, in this case, a nucleic acid indicative of syphilis infection. The term “negative,” when referring to a result or signal, indicates the absence of an analyte or item that is being detected in a sample. Positive and negative can be determined by comparison to at least one control, e.g., a threshold level for a sample to be determined positive, or a negative control (e.g., a known blank).

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters, and will understand which controls are valuable in a given situation and be able to analyze data based on comparisons to control values. Controls are also valuable for determining the significance of data. For example, if values for a given parameter are variable in controls, variation in test samples will not be considered as significant.

As used herein, “percent identity”, as indicated by a percentage and the word “identity” (e.g., “80% identity”), refers to the extent to which a polynucleotide sequence is the same as a reference polynucleotide sequence when the sequences are optimally aligned. The sequences can be aligned using known pairwise sequence alignment tools and procedures. Once the sequences are optimally aligned, bases at corresponding positions within the alignment that are the same within both sequences are counted to yield a total of common bases, which are expressed as a percentage of the total bases within the alignment. For example, if an example sequence is ATGTGAGGTC and a reference sequence is ATGTGAAATC, then an optimal alignment of these sequences would reveal that 8 out of 10 nucleotide bases in the example sequence are identical with the corresponding bases in the reference sequence, and therefore the example sequence has 80% identity to the reference sequence.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

Unless the context clearly requires otherwise, the phrase “consisting essentially of” limits the scope of a claim to the specified materials or steps and those that do not materially affect the basic and novel characteristic(s) of the claim.

Unless the context clearly requires otherwise, the phrase “consisting of” excludes any element, step, or ingredient not specified.

If an element is described or claimed herein such that it “comprises” a feature, that description or claim also includes embodiments wherein the element “consists essentially of” and embodiments wherein the element “consists of” the feature, unless something else is specifically stated to the contrary.

Words using the singular or plural number also include the plural and singular number, respectively. Additionally, the words “herein,” “above,” and “below” and words of similar import, when used in this application, shall refer to this application as a whole and not to any particular portions of the application.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

It will be appreciated that, although specific embodiments of the disclosure have been described herein for purposes of illustration, various modifications may be made without deviating from the spirit and scope of the disclosure. Accordingly, the disclosure is not limited except as stated by the claims.

### Non-Limiting Embodiments

While general features of the disclosure are described and shown and particular features of the disclosure are set forth in the claims, the following non-limiting embodiments relate to features, and combinations of features, that are explicitly envisioned as being part of the disclosure. The following non-limiting Embodiments contain elements that are modular and can be combined with each other in any number, order, or combination to form a new non-limiting Embodiment, which can itself be further combined with other non-limiting Embodiments.

Embodiment 1. A site-specific primer comprising polynucleotide sequence configured to anneal to a T. pallidum nucleic acid for a loop-mediated isothermal amplification (LAMP) reaction.

Embodiment 2. A composition for detection of a portion of a T. pallidum nucleic acid in a sample, the composition comprising the site-specific primer of Embodiment 1.

Embodiment 3. The composition of Embodiment 2, further comprising: a plurality of site-specific primers comprising polynucleotide sequences configured to anneal to the T. pallidum nucleic acid in the sample, wherein the site-specific primer is of the plurality of site-specific primers.

Embodiment 4. The composition of any one of Embodiments 2-3, further comprising: a strand-displacing DNA polymerase configured to amplify the portion of the T. pallidum nucleic acid based on anneal positions of the plurality of site-specific primers to produce amplified products of the LAMP reaction; an intercalating agent, a probe, or a fluorophore for detection of the portion of the T. pallidum nucleic acid; or any combination thereof.

Embodiment 5. The composition of any one of Embodiments 3-4, wherein the plurality of site-specific primers comprises a forward inner primer (FIP), a backward inner primer (BIP), a forward outer primer (FOP), and a backward outer primer (BOP).

Embodiment 6. The composition of any one of Embodiments 3-5, wherein the plurality of site-specific primers further comprises a forward loop primer (FLP), a backward loop primer (BLP), or both.

Embodiment 7. The composition of any one of Embodiments 2-6, wherein the portion of the T. pallidum nucleic acid comprises at least a portion of a D1 domain of a 23S rRNA gene of the T. pallidum genome.

Embodiment 8. The site-specific primer of Embodiment 1 or the composition of any one of Embodiments 2-7, wherein a sequence of the site-specific primer has at least 80% identity to a sequence selected from SEQ ID NOs: 1-12.

Embodiment 9. The site-specific primer of Embodiment 1 or the composition of any one of Embodiments 2-7, wherein a sequence of the site-specific primer has at least 90% identity to a sequence selected from SEQ ID NOs: 1-12.

Embodiment 10. The site-specific primer of Embodiment 1 or the composition of any one of Embodiments 2-7, wherein a sequence of the site-specific primer has 100% identity to a sequence selected from SEQ ID NOs: 1-12.

Embodiment 11. The composition of any one of Embodiments 3-10, wherein: a forward inner primer (FIP) of the plurality of site-specific primers comprises SEQ ID NO: 3 or SEQ ID NO 9; a backward inner primer (BIP) of the plurality of site-specific primers comprises SEQ ID NO: 4 or SEQ ID NO: 10; a forward outer primer (FOP) of the plurality of site-specific primers comprises SEQ ID NO: 1 or SEQ ID NO: 7; a backward outer primer (BOP) of the plurality of site-specific primers comprises SEQ ID NO: 2 or SEQ ID NO: 8; a forward loop primer (FLP) of the plurality of site-specific primers comprises SEQ ID NO: 5 or SEQ ID NO: 11; and a backward loop primer (BLP) of the plurality of site-specific primers comprises SEQ ID NO: 6 or SEQ ID NO: 12.

Embodiment 12. A kit for detection of a portion of a T. pallidum nucleic acid in a sample, the kit comprising the site-specific primer of Embodiment 1 or the composition of any one of Embodiments 2-11.

Embodiment 13. The kit of Embodiment 12, further comprising: an instructional material for use of the kit in a method for detection of syphilis in the patient.

Embodiment 14. A loop-mediated isothermal amplification (LAMP) reaction method for detection of a portion of a T. pallidum nucleic acid in a sample, the method comprising: contacting the sample with the site-specific primer of Embodiment 1 or the composition of any of Embodiments 2-11 and enabling the LAMP reaction to occur; and detecting the portion of the T. pallidum nucleic acid in amplified products of the LAMP reaction.

Embodiment 15. The method of Embodiment 14, wherein the detecting the portion of the T. pallidum nucleic acid in amplified products of the LAMP reaction comprises: contacting the sample with an intercalating agent, a probe, or a fluorophore for fluorescence detection of the portion of the T. pallidum nucleic acid; performing a LFA for detection of the portion of the T. pallidum nucleic acid; performing agarose gel electrophoresis for detection of the portion of the T. pallidum nucleic acid; or any combination thereof.

Embodiment 16. The method of any one of Embodiments 14-15, wherein the sample is a clinical sample and the method is a point-of-care (POC) method.

Embodiment 17. The method of any one of Embodiments 14-16, wherein the sample comprises a swab sample, a cellular tissue specimen, or a body fluid.

Embodiment 18. The method of Embodiment 17, wherein the sample comprises the body fluid and the body fluid comprises whole blood, serum, plasma, peripheral blood mononuclear cells (PBMCs), a non-bloody body fluid, cerebrospinal fluid (CSF), or amniotic fluid.

Embodiment 19. The method of any one of Embodiments 14-18, wherein the method has a limit of detection (LOD) of about 7-10 copies of the portion of the T. pallidum nucleic acid.

Embodiment 20. The method of any one of Embodiments 14-19, wherein the method has specificity for detection of T. pallidum nucleic acids.

Embodiment 21. The method of any one of Embodiments 14-20, wherein T. pallidum includes T. pallidum subspecies pertenue, T. pallidum subspecies endemicum, or T. pallidum subspecies pallidum.

Embodiment 22. The method of Embodiment 21, wherein T. pallidum includes T. pallidum subspecies pallidum and the method is for detection of syphilis in a patient.

While illustrative embodiments have been illustrated and described, it will be appreciated that various changes can be made therein without departing from the spirit and scope of the disclosure.

1. A site-specific primer comprising polynucleotide sequence configured to anneal to a T. pallidum nucleic acid for a loop-mediated isothermal amplification (LAMP) reaction.

2. A composition for detection of a portion of a T. pallidum nucleic acid in a sample, the composition comprising the site-specific primer of claim 1.

3. The composition of claim 2, further comprising:
a plurality of site-specific primers comprising polynucleotide sequences configured to anneal to the T. pallidum nucleic acid in the sample, wherein the site-specific primer is of the plurality of site-specific primers.

4. The composition of claim 2, further comprising:
a strand-displacing DNA polymerase configured to amplify the portion of the T. pallidum nucleic acid based on anneal positions of the plurality of site-specific primers to produce amplified products of the LAMP reaction;
an intercalating agent, a probe, or a fluorophore for detection of the portion of the T. pallidum nucleic acid; or
any combination thereof.

5. The composition of claim 3, wherein the plurality of site-specific primers comprises a forward inner primer (FIP), a backward inner primer (BIP), a forward outer primer (FOP), and a backward outer primer (BOP).

6. The composition of claim 3, wherein the plurality of site-specific primers further comprises a forward loop primer (FLP), a backward loop primer (BLP), or both.

7. The composition of claim 2, wherein the portion of the T. pallidum nucleic acid comprises at least a portion of a D1 domain of a 23S rRNA gene of the T. pallidum genome.

8-9. (canceled)

10. The composition of claim 2, wherein a sequence of the site-specific primer has at least 80% identity, at least 90% identity, or 100% identity to a sequence selected from SEQ ID NOs: 1-12.

11. The composition of claim 3, wherein:
a forward inner primer (FIP) of the plurality of site-specific primers comprises SEQ ID NO: 3 or SEQ ID NO 9;
a backward inner primer (BIP) of the plurality of site-specific primers comprises SEQ ID NO: 4 or SEQ ID NO: 10;
a forward outer primer (FOP) of the plurality of site-specific primers comprises SEQ ID NO: 1 or SEQ ID NO: 7;
a backward outer primer (BOP) of the plurality of site-specific primers comprises SEQ ID NO: 2 or SEQ ID NO: 8;
a forward loop primer (FLP) of the plurality of site-specific primers comprises SEQ ID NO: 5 or SEQ ID NO: 11; and
a backward loop primer (BLP) of the plurality of site-specific primers comprises SEQ ID NO: 6 or SEQ ID NO: 12.

12. A kit for detection of a portion of a T. pallidum nucleic acid in a sample, the kit comprising the site-specific primer of claim 1.

13. The kit of claim 12, further comprising:
an instructional material for use of the kit in a method for detection of syphilis in the patient.

14. A loop-mediated isothermal amplification (LAMP) reaction method for detection of a portion of a T. pallidum nucleic acid in a sample, the method comprising:
contacting the sample with the site-specific primer of claim 1 and enabling the LAMP reaction to occur; and
detecting the portion of the T. pallidum nucleic acid in amplified products of the LAMP reaction.

15. The method of claim 14, wherein the detecting the portion of the T. pallidum nucleic acid in amplified products of the LAMP reaction comprises:
contacting the sample with an intercalating agent, a probe, or a fluorophore for fluorescence detection of the portion of the T. pallidum nucleic acid;
performing a LFA for detection of the portion of the T. pallidum nucleic acid;
performing agarose gel electrophoresis for detection of the portion of the T. pallidum nucleic acid; or
any combination thereof.

16. The method of claim 14, wherein the sample is a clinical sample and the method is a point-of-care (POC) method.

17. The method of claim 14, wherein the sample comprises a swab sample, a cellular tissue specimen, or a body fluid.

18. The method of claim 17, wherein the sample comprises the body fluid and the body fluid comprises whole blood, serum, plasma, peripheral blood mononuclear cells (PBMCs), a non-bloody body fluid, cerebrospinal fluid (CSF), or amniotic fluid.

19. The method of claim 14, wherein the method has a limit of detection (LOD) of about 7-10 copies of the portion of the T. pallidum nucleic acid.

20. The method of claim 14, wherein the method has specificity for detection of T. pallidum nucleic acids.

21. The method of claim 14, wherein T. pallidum includes T. pallidum subspecies pertenue, T. pallidum subspecies endemicum, or T. pallidum subspecies pallidum.

22. The method of claim 21, wherein T. pallidum includes T. pallidum subspecies pallidum and the method is for detection of syphilis in a patient.


SEQUENCE LISTING (64 sequences)

SEQ ID NO: 1 - DNA, 18 bp, synthetic construct
  source 1..18: mol_type = other DNA
aaaagcctcggggaggag

SEQ ID NO: 2 - DNA, 20 bp, synthetic construct
  source 1..20: mol_type = other DNA
agacacaggtttaggctcct

SEQ ID NO: 3 - DNA, 40 bp, synthetic construct
  source 1..40: mol_type = other DNA
tcaggaaggcaatgacaaggcttcctgtgatccggggatg

SEQ ID NO: 4 - DNA, 41 bp, synthetic construct
  source 1..41: mol_type = other DNA
aaggcgaaactgggtgaactgattcgctcgccactactttc

SEQ ID NO: 5 - DNA, 20 bp, synthetic construct
  source 1..20: mol_type = other DNA
ctgtcgggttaccccattcg

SEQ ID NO: 6 - DNA, 25 bp, synthetic construct
  source 1..25: mol_type = other DNA
gggaaaagaaatcaagagagattcc

SEQ ID NO: 7 - DNA, 19 bp, synthetic construct
  source 1..19: mol_type = other DNA
aggcgatgaaggtcgtgat

SEQ ID NO: 8 - DNA, 20 bp, synthetic construct
  source 1..20: mol_type = other DNA
acaggtttaggctcctccaa

SEQ ID NO: 9 - DNA, 40 bp, synthetic construct
  source 1..40: mol_type = other DNA
gctttaccctgtcgggttacccgggaggagcacatgtcct

SEQ ID NO: 10 - DNA, 41 bp, synthetic construct; identical to SEQ ID NO: 4
  source 1..41: mol_type = other DNA

SEQ ID NO: 11 - DNA, 17 bp, synthetic construct
  source 1..17: mol_type = other DNA
ggtcatccccggatcac

SEQ ID NO: 12 - DNA, 25 bp, synthetic construct
  source 1..25: mol_type = other DNA
gggaaaagaaatcaagagagattcc

SEQ ID NO: 13 - DNA, 270 bp, Treponema pallidum
  source 1..270: mol_type = genomic DNA
gtttacggtggatgtcttggagttgtcaggcgatgaaggtcgtgataagctgcgaaaagcctcggggaggagcacatgtcctgtgatccggggatgaccgaatggggtaacccgacagggtaaagccttgtcattgccttcctgaatgaatagggagggtaaggcgaaactgggtgaactgaaccatctaagtaacttgggaaaagaaatcaagagagattccgaaagtagtggcgagcgaaattggaggagcctaaacctgtgtctaac

SEQ ID NO: 14 - DNA, 106 bp, Treponema pallidum
  source 1..106: mol_type = other DNA
gccttgtcattgccttcctgaatgaatagggagggtaaggcgaaactgggtgaactgaaccatctaagtaacttgggaaaagaaatcaagagagattccgaaagta

SEQ ID NO: 15 - DNA, 106 bp, Treponema pallidum; identical to SEQ ID NO: 14
  source 1..106: mol_type = other DNA

SEQ ID NO: 16 - DNA, 106 bp, Treponema pallidum; identical to SEQ ID NO: 14
  source 1..106: mol_type = other DNA

SEQ ID NO: 17 - DNA, 106 bp, Treponema pallidum; identical to SEQ ID NO: 14
  source 1..106: mol_type = other DNA

SEQ ID NO: 18 - DNA, 106 bp, Treponema pallidum; identical to SEQ ID NO: 14
  source 1..106: mol_type = other DNA

SEQ ID NO: 19 - DNA, 106 bp, Treponema pallidum; identical to SEQ ID NO: 14
  source 1..106: mol_type = other DNA

SEQ ID NO: 20 - DNA, 46 bp, Treponema pallidum
  source 1..46: mol_type = other DNA
gccttgtcattgccttcctgaatgaatagggagggtaaggcgaaac

SEQ ID NO: 21 - DNA, 46 bp, Treponema pallidum; identical to SEQ ID NO: 20
  source 1..46: mol_type = other DNA

SEQ ID NO: 22 - DNA, 46 bp, Treponema pallidum; identical to SEQ ID NO: 20
  source 1..46: mol_type = other DNA

SEQ ID NO: 23 - DNA, 46 bp, Treponema pallidum; identical to SEQ ID NO: 20
  source 1..46: mol_type = other DNA

SEQ ID NO: 24 - DNA, 46 bp, Treponema pallidum; identical to SEQ ID NO: 20
  source 1..46: mol_type = other DNA

SEQ ID NO: 25 - DNA, 46 bp, Treponema pallidum; identical to SEQ ID NO: 20
  source 1..46: mol_type = other DNA

SEQ ID NO: 26 - DNA, 46 bp, Treponema pallidum; identical to SEQ ID NO: 20
  source 1..46: mol_type = other DNA

SEQ ID NO: 27 - DNA, 46 bp, Treponema pallidum; identical to SEQ ID NO: 20
  source 1..46: mol_type = other DNA

SEQ ID NO: 28 - DNA, 118 bp, Treponema pallidum
  source 1..118: mol_type = other DNA
gccttgtcattgccttcctgaatgaatagggagggtaaggcgaaactgggtgaactgaaccatctaagtaacttgggaaaagaaatcaagagagattccgaaagtagtggcgagcgaa

SEQ ID NO: 29 - DNA, 118 bp, synthetic construct
  source 1..118: mol_type = other DNA
gccttgtcattgccttcctgaatgttatgggagggtaaggcgaaactgggtgaactgaacctagattcattcttgggaaaagaaatcaagagagattccgaaagtagtggcgagcgaa

SEQ ID NO: 30 - DNA, 118 bp, synthetic construct; identical to SEQ ID NO: 29
  source 1..118: mol_type = other DNA

SEQ ID NO: 31 - DNA, 118 bp, synthetic construct; identical to SEQ ID NO: 29
  source 1..118: mol_type = other DNA

SEQ ID NO: 32 - DNA, 118 bp, synthetic construct; identical to SEQ ID NO: 29
  source 1..118: mol_type = other DNA

SEQ ID NO: 33 - DNA, 118 bp, synthetic construct; identical to SEQ ID NO: 29
  source 1..118: mol_type = other DNA

SEQ ID NO: 34 - DNA, 73 bp, synthetic construct
  source 1..73: mol_type = other DNA
gccttgtcattgccttcctgaatgttatgggagggtaaggcgaaactgggtgaactgaacctagattcattct

SEQ ID NO: 35 - DNA, 60 bp, synthetic construct
  source 1..60: mol_type = other DNA
gccttgtcattgccttcctgaatgaaaccctgggtaaggcgaaactgggtgaactgacct

SEQ ID NO: 36 - DNA, 31 bp, Treponema pallidum
  source 1..31: mol_type = other DNA
aatgaatagggagggtaccatctaagtaact

SEQ ID NO: 37 - DNA, 31 bp, Treponema pallidum; identical to SEQ ID NO: 36
  source 1..31: mol_type = other DNA

SEQ ID NO: 38 - DNA, 31 bp, Treponema pallidum; identical to SEQ ID NO: 36
  source 1..31: mol_type = other DNA

SEQ ID NO: 39 - DNA, 31 bp, Treponema pallidum; identical to SEQ ID NO: 36
  source 1..31: mol_type = other DNA

SEQ ID NO: 40 - DNA, 31 bp, Treponema pallidum; identical to SEQ ID NO: 36
  source 1..31: mol_type = other DNA

SEQ ID NO: 41 - DNA, 31 bp, Treponema pallidum; identical to SEQ ID NO: 36
  source 1..31: mol_type = other DNA

SEQ ID NO: 42 - DNA, 31 bp, Treponema pallidum; identical to SEQ ID NO: 36
  source 1..31: mol_type = other DNA

SEQ ID NO: 43 - DNA, 31 bp, synthetic construct
  source 1..31: mol_type = other DNA
aatgttatccctcggtacctagattcattct

SEQ ID NO: 44 - DNA, 31 bp, synthetic construct; identical to SEQ ID NO: 43
  source 1..31: mol_type = other DNA

SEQ ID NO: 45 - DNA, 31 bp, synthetic construct; identical to SEQ ID NO: 43
  source 1..31: mol_type = other DNA

SEQ ID NO: 46 - DNA, 31 bp, synthetic construct; identical to SEQ ID NO: 43
  source 1..31: mol_type = other DNA

SEQ ID NO: 47 - DNA, 31 bp, synthetic construct; identical to SEQ ID NO: 43
  source 1..31: mol_type = other DNA

SEQ ID NO: 48 - DNA, 31 bp, synthetic construct; identical to SEQ ID NO: 43
  source 1..31: mol_type = other DNA

SEQ ID NO: 49 - DNA, 19 bp, synthetic construct
  source 1..19: mol_type = other DNA
aatgtttccctcggtacct

SEQ ID NO: 50 - DNA, 125 bp, Treponema pallidum
  source 1..125: mol_type = other DNA
gggtaaagccttgtcattgccttcctgaatgaatagggagggtaaggcgaaactgggtgaactgaaccatctaagtaacttgggaaaagaaatcaagagagattccgaaagtagtggcgagcgaa

SEQ ID NO: 51 - DNA, 240 bp, unidentified
  source 1..240: mol_type = other DNA
gcactctgcaagttttcccatcacatttttaggctccttacctgcttttaatccccggatccaggagccttctcgggggtcaggcgggggaatgtgtccgggtttctatggggaaaaaaaagggtggggccttgtcattgccttcctgaatgatttgggtgggtaaggcgaaactgggtggttttttcggaatcaaagaaattttttttttttaaattcgccgtttttttgatgtccagg

SEQ ID NO: 52 - DNA, 104 bp, Treponema pallidum
  source 1..104: mol_type = other DNA
aagccttgtcattgccttcctgaatgaatagggagggtaaggcgaaactgggtgaactgaaccatctaagtaacttgggaaaagaaatcaagagagattccgaa

SEQ ID NO: 53 - DNA, 104 bp, Treponema pallidum; identical to SEQ ID NO: 52
  source 1..104: mol_type = other DNA

SEQ ID NO: 54 - DNA, 104 bp, Treponema pallidum; identical to SEQ ID NO: 52
  source 1..104: mol_type = other DNA

SEQ ID NO: 55 - DNA, 104 bp, Treponema pallidum; identical to SEQ ID NO: 52
  source 1..104: mol_type = other DNA

SEQ ID NO: 56 - DNA, 104 bp, Treponema pallidum
  source 1..104: mol_type = other DNA
aagccttgtcattgccttcctgaatgttatccctgggtaaggcgaaactgggtgaactgaaccatctaagtaacttgggaaaagaaatcaagagagattccgaa

SEQ ID NO: 57 - DNA, 18 bp, synthetic construct
  source 1..18: mol_type = other DNA
tcctgtgatccggggatg

SEQ ID NO: 58 - DNA, 22 bp, synthetic construct
  source 1..22: mol_type = other DNA
agccttgtcattgccttcctga

SEQ ID NO: 59 - DNA, 22 bp, synthetic construct
  source 1..22: mol_type = other DNA
aaggcgaaactgggtgaactga

SEQ ID NO: 60 - DNA, 19 bp, synthetic construct
  source 1..19: mol_type = other DNA
gaaagtagtggcgagcgaa

SEQ ID NO: 61 - DNA, 18 bp, synthetic construct
  source 1..18: mol_type = other DNA
tcctgtgatccggggatg

SEQ ID NO: 62 - DNA, 19 bp, synthetic construct
  source 1..19: mol_type = other DNA
ttcgctcgccactactttc

SEQ ID NO: 63 - DNA, 125 bp, synthetic construct
  source 1..125: mol_type = other DNA
ggcataagccttgtcattgccttcctgaatgttatccctcggtaaggcgaaactgggtgaactgaacctagattcattcttgggaaaagaaatcaagagagattccgaaagtagtggcgagcgaa

SEQ ID NO: 64 - DNA, 500 bp, synthetic construct
  source 1..500: mol_type = other DNA
gtttacggtggatgtcttggagttgtcaggcgatgaaggtcgtgataagctgcgaaaagcctcggggaggagcacatgtcctgtgatccggggatgaccgaatggggtaacccgacaggcataagccttgtcattgccttcctgaatgttatccctcggtaaggcgaaactgggtgaactgaacctagattcattcttgggaaaagaaatcaagagagattccgaaagtagtggcgagcgaaattggaggagcctaaacctgtgtctaacaggggttgtagggccgcgcgggcttgcgttcggtgggtgaaataatccggcctatagcagaaaggttttgggaaagcctgacagagagggtgaaatccccgtatgcggaatggggcggacctgctggtgcggtacctgagtacggcgggacacgaggaatcctgtcggaatctgggtcgaccacgatctaaggctaaatactcgacaactaccgatagtggacaagtacc